(19) United States
(12) Patent Application Publication
(10) Pub. No.: US 2026/0132437 A1
(43) Pub. Date: May 14, 2026

(54) Methods of Synthesizing Nucleic Acid Molecules

(71) Applicant: Telesis Bio Inc, San Diego, CA (US)

(72) Inventors: John E Gill, San Diego, CA (US); Lixia Fu, San Diego, CA (US); Sydney Kerr, San Marcos, CA (US); MIchelle Vargas, San Diego, CA (US); Daniel G Gibson, Carlsbad, CA (US)

(73) Assignee: Telesis Bio Inc, San Diego, CA (US)

(21) Appl. No.: 19/121,294

(22) PCT Filed: Oct. 31, 2022
(86) PCT No.: PCT/US2022/048407

§ 371 Date: Apr. 15, 2025

(51) Int. Cl. C12P 19/34; C12N 9/00; C12N 9/12; C12N 9/16; C12N 15/10

(52) U.S. Cl. CPC C12P 19/34; C12N 9/1252; C12N 9/16; C12N 9/93; C12N 15/1068; C12Y 207/07007; C12Y 301/21004; C12Y 605/01001

(57) ABSTRACT

The invention provides methods for synthesizing a product DNA molecule of any DNA sequence from a universal library of overlapping oligonucleotides. The method involves combining a plurality of the overlapping oligonucleotides in a reaction pool, where the sequences of the plurality of oligonucleotides comprise at least a sub-sequence of the product DNA molecule. The method also involves annealing the plurality of oligonucleotides, performing a ligation step, and performing an amplification step to thereby synthesize a sub-sequence of the product DNA molecule. The invention can be used to synthesize a DNA molecule of any possible sequence from the universal library, which can be accomplished through a hierarchal assembly scheme. In one embodiment the universal library comprises fewer than 10,000 pre-manufactured oligonucleotides that can be synthesized into the any possible DNA sequence.

### FIELD OF THE INVENTION

The invention provides compositions, methods, and kits for synthesizing any possible DNA molecule from a limited library of oligonucleotides.

### BACKGROUND OF THE INVENTION

The fields of synthetic biology and gene editing and therapeutics have a continuing and growing need for oligonucleotides of diverse and known sequences. Existing methods for synthesizing small oligonucleotides involve chemical synthesis via solid-phase, sequential coupling of nucleotides to generate the oligonucleotide of desired length and sequence. Oligonucleotides produced are then released from the solid phase, deprotected, and collected for assembly into larger oligonucleotides by other methods. While automated, these processes are subject to side reactions and base errors, thus limiting the length of the oligonucleotides produced. For applications requiring ultra-high sequence fidelity, these methods have additional limitations.

Enzymatic methods of synthesizing oligonucleotides also exist and involve the use of enzymes such as terminal deoxynucleotidyl transferase (TdT), a template-independent polymerase that catalyzes the incorporation of deoxyribonucleotides into the 3′-hydroxyl end of DNA templates. But the enzyme shows strong bias for specific nucleotide bases and does not reliably add nucleotides in the desired order and length.

There is a continuing need for methods of synthesizing oligonucleotides efficiently and with high fidelity so that the user can produce oligonucleotides of any desired length and sequence.

### SUMMARY OF THE INVENTION

The invention provides methods for synthesizing a product DNA molecule of any possible DNA sequence from a library of overlapping oligonucleotides, which can be a universal library. The method involves combining a plurality of the overlapping oligonucleotides in a reaction pool, where the sequences of the plurality of oligonucleotides comprise at least a sub-sequence of the product DNA molecule. The method also involves annealing the plurality of oligonucleotides, performing a ligation step, and performing an amplification step to thereby synthesize a sub-sequence of the product DNA molecule. The invention can be used to synthesize a DNA molecule of any possible sequence from the library, which can be accomplished through a hierarchal assembly scheme. In one embodiment the library is a universal library having fewer than 10,000 pre-manufactured oligonucleotides, which can be synthesized into the any possible DNA sequence using the methods. The product DNA molecule can be at least 100 base pairs or at least 150 base pairs long. When subsequent DNA assembly techniques are employed DNA molecules of thousands of base pairs can be synthesized. In any embodiment the product DNA molecule can have an error rate of less than 1 error per 2,000 nucleotides.

In a first aspect the invention provides methods of synthesizing a DNA molecule having a desired sequence. The methods involve annealing at least two oligonucleotides to an anchor strand so that the at least two oligonucleotides annealed to the anchor strand abut one another on the anchor strand. In any embodiment the oligonucleotides can abut on the anchor strand at their variable sequences. The at least two oligonucleotides can each comprise a primer binding site on a 3′ or 5′ end, and a variable sequence on the opposing 5′ or 3′ end, and a conserved flanking sequence in between the primer binding site and the variable sequence. The anchor strand can have conserved flanking sequences complementary to those on the at least two oligonucleotides, and further can have at least one variable sequence. At least one portion of the at least one variable sequence on the anchor strand is complementary to at least a portion of the variable sequence on each of the at least two oligonucleotides. The invention involves a step of ligating the at least two oligonucleotides annealed to the anchor strand to produce a first dsDNA molecule, performing an amplification step on the first dsDNA molecule having a desired sequence and comprising primer binding sites at the 3′ and 5′ ends, a conserved flanking sequence inside each of the 3′ and 5′ ends, and a variable sequence inside the conserved flanking sequences. In one embodiment the first dsDNA molecule has a variable sequence that is about 8 or about 10 nucleotides long. In any embodiment one or more (or all) of the primer binding sites can be universal primer binding sites.

The method can also involve contacting the first dsDNA molecule with a restriction endonuclease to produce first dsDNA fragments having 3′ and/or 5′ overhang sequences comprising a portion of the variable sequence from the first dsDNA molecule, providing at least one additional dsDNA fragment comprising a 3′ and/or 5′ overhang sequence that is at least partially complementary to an overhang sequence of at least one of the first dsDNA fragments. The 3′ and/or 5′ overhang sequences can contain at least a portion of the variable sequence. The methods also involve annealing the first dsDNA fragments and at least one additional dsDNA fragment by the 3′ and/or 5′ overhang sequences, and ligating the annealed dsDNA fragments to produce a second dsDNA molecule having a conserved flanking sequence inside each of the 3′ and 5′ ends, and a variable sequence inside the 3′ and 5′ conserved flanking sequences that is longer than the variable sequence on the first dsDNA molecule (and optional primer binding sites on the 3′ and/or 5′ ends). In one embodiment the variable sequence of the second dsDNA molecule is about 16 base pairs in length. The method can also involve performing an amplification step on the second dsDNA molecule. In any embodiment the restriction endonuclease can be a Type II restriction endonuclease (e.g. Type IIS or Type IIT). In any embodiment and any step of the methods the at least one additional dsDNA fragment can be the product of a parallel DNA synthesis reaction. The first dsDNA molecule can have a recognition site for a restriction endonuclease on the 5′ or 3′ side of the molecule, and the first additional dsDNA fragment can be derived from restriction cleavage of a dsDNA molecule having a recognition site for a restriction endonuclease on the opposing 3′ or 5′ side of the molecule.

The method can further involve contacting the at least one second dsDNA molecule with a restriction endonuclease to produce a plurality of second dsDNA fragments comprising 3′ and/or 5′ overhang sequences and a conserved flanking sequence inside each of the 3′ or 5′ ends (and optional primer binding sites on the 3′ and/or 5′ ends). The 3′ and/or 5′ overhang sequences can contain at least a portion of the variable sequence. The method can further involve a step of providing at least one (second) additional dsDNA fragment comprising a 3′ and/or 5′ overhang sequence that is at least partially complementary to an overhang sequence of at least one of the second dsDNA fragments, annealing the plurality of second dsDNA fragments to the one or more (second) additional dsDNA fragment(s) by the 3′ and/or 5′ overhang sequence(s); and performing a step of ligation to produce a third dsDNA molecule having a conserved flanking sequence on the 3′ and 5′ ends, and a variable sequence inside the conserved flanking sequences that is longer than the variable sequence of the second dsDNA molecule (and optional primer binding sites on the 3′ and/or 5′ ends). In one embodiment the variable sequence is about 28 base pairs long. The method can include performing an amplification step on the third dsDNA molecule. The at least one (second) additional dsDNA fragment can be the product of a parallel DNA synthesis reaction. The second dsDNA molecule can have a recognition site for a restriction endonuclease on the 5′ or 3′ side of the molecule, and the (second) additional dsDNA fragment can be derived from restriction cleavage of a dsDNA molecule having a recognition site for a restriction endonuclease on the opposing 3′ or 5′ side of the molecule.

The method can further involve contacting the at least one third dsDNA molecule with a restriction endonuclease to produce a plurality of third dsDNA fragments comprising 3′ and/or 5′ overhang sequences and a conserved flanking sequence inside each of the 3′ or 5′ ends (and optional primer binding sites on the 3′ and/or 5′ ends); the fragments can contain at least a portion of the variable sequence on the 3′ and/or 5′ overhangs. The methods can also involve providing at least one (third) additional dsDNA fragment comprising a 3′ and/or 5′ overhang sequence that is at least partially complementary to an overhang sequence of at least one of the third dsDNA fragments. The overhang sequences can contain at least a portion of the variable sequence. The methods can also involve annealing the plurality of third dsDNA fragments to the one or more (third) additional dsDNA fragment(s) by the 3′ and/or 5′ overhang sequence(s). The method can further involve performing a step of ligation to produce a fourth dsDNA molecule having a conserved flanking sequence on the 3′ and 5′ ends, and a variable sequence inside the conserved flanking sequences that is longer than the variable sequence of the third dsDNA molecule (and optional primer binding sites on the 3′ and/or 5′ ends). The (third) additional dsDNA fragment can be the product of a parallel DNA synthesis reaction. The third dsDNA molecule can have a recognition site for a restriction endonuclease on the 5′ or 3′ side of the molecule, and the (third) additional dsDNA fragment can be derived from restriction cleavage of a dsDNA molecule having a recognition site for a restriction endonuclease on the opposing 3′ or 5′ side of the molecule. The methods can also involve performing an amplification step on the fourth dsDNA molecule. In one embodiment the variable sequence is about 100 base pairs long.

In another embodiment step a) further involves annealing at least two paired oligonucleotides to a paired anchor strand so that the at least two paired oligonucleotides bound to the paired anchor strand abut one another on the paired anchor strand, which can occur at their variable sequences. The at least two paired oligonucleotides can have a primer binding site on a 3′ or 5′ end, and a variable sequence on the opposing 5′ or 3′ end, and a conserved flanking sequence in between the primer binding site and the variable sequence. The paired anchor strand can have conserved flanking sequences complementary to those on the at least two paired oligonucleotides, and further have at least one variable sequence. A portion of the variable sequence on the paired anchor strand can overlap with a portion of the variable sequence on the first anchor strand. The variable sequence can be located in between the two sequences complementary to the conserved flanking sequences. At least a portion of the variable sequence differs between the first and second anchor strands. In any embodiment one or more (or all) of the primer binding sites can be universal primer binding sites.

The method can further involve ligating the at least two paired oligonucleotides annealed to the anchor strand, performing an amplification step to produce a paired dsDNA molecule of desired sequence and comprising a primer binding site at a 3′ and 5′ end, a conserved flanking sequence inside each of the 3′ and 5′ ends, and a variable sequence inside the conserved flanking sequences that partially overlaps with the variable sequence of the first dsDNA molecule. In one embodiment the at least two oligonucleotides and first anchor strand, and the at least two paired oligonucleotides and paired anchor strand can be annealed in a simultaneous reaction in the same pool. The method can further involve contacting the first dsDNA molecule and the paired dsDNA molecule with a restriction endonuclease to produce at least one dsDNA fragment and at least one paired dsDNA fragment, each comprising at least one 3′ and/or 5′ overhang sequence; and at least a portion of a 3′ or 5′ overhang sequence from a first dsDNA fragment can be complementary to at least a portion of a 5′ or 3′ overhang sequence from a paired dsDNA fragment (and optional primer binding sites on the 3′ or 5′ end). The method can further involve annealing the first and paired dsDNA fragments by their complementary overhang sequences and performing a step of ligation to produce a second dsDNA molecule having a conserved flanking sequence inside each of the 3′ and 5′ ends, and a variable sequence inside the 3′ and 5′ conserved flanking sequences that is longer than the variable sequence on the first dsDNA molecules (and optional primer binding sites on the 3′ and/or 5′ ends). The method can also involve performing an amplification step on the second dsDNA molecule.

In a further embodiment the method can further involve contacting the at least one second dsDNA molecule and an at least one paired second dsDNA molecule with a restriction endonuclease to produce a plurality of second dsDNA fragments and paired second dsDNA fragments, each comprising a 3′ and/or 5′ overhang sequence(s). At least two of the plurality comprise, a conserved flanking sequence inside each of the 3′ or 5′ ends. At least a portion of the 3′ or 5′ overhang sequence from a second dsDNA fragment can be complementary to at least a portion of the 5′ or 3′ overhang sequence from a paired second dsDNA fragment. The method can further involve annealing the second and paired second in dsDNA fragments by their complementary overhang sequences, and performing a step of ligation to produce a third dsDNA molecule comprising a conserved flanking sequence inside each of the 3′ and 5′ ends, and a variable sequence inside the 3′ and 5′ conserved flanking sequences that is longer than the variable sequence on the second dsDNA molecules (and optional primer binding sites on the 3′ and/or 5′ ends). At least a portion of the variable sequence on the third dsDNA molecule can overlap with at least a portion of the variable sequence on the paired third dsDNA molecule. The methods can also involve performing a step of amplification on the third dsDNA molecule.

In another embodiment the methods further involve contacting the at least one third dsDNA molecule and an at least one paired third dsDNA molecule with a restriction endonuclease to produce a plurality of third dsDNA fragments and paired third dsDNA fragments, each comprising a 3′ and/or 5′ overhang sequence(s); the third dsDNA fragments can have at least a portion of the variable sequence on the 3′ and/or 5′ overhangs. At least two of the plurality can have a conserved flanking sequence inside the 3′ or 5′ ends. At least a portion of the 3′ or 5′ overhang sequence from a third dsDNA fragment can be complementary to at least a portion of the 5′ or 3′ overhang sequence from a paired third dsDNA fragment. The methods can further involve a step of annealing the third and paired third dsDNA fragments by their complementary overhang sequences, and performing a step of ligation to produce a fourth dsDNA molecule having a conserved flanking sequence inside each of the 3′ and 5′ ends, and a variable sequence inside the 3′ and 5′ conserved flanking sequences that is longer than the variable sequence on the third dsDNA molecule (and optional primer binding sites on the 3′ and/or 5′ ends). The methods can also involve performing a step of amplification on the fourth dsDNA molecule.

In any embodiment the first dsDNA molecule can have a variable region of 8-12 base pairs. In any embodiment the paired dsDNA molecule can have a variable region of 8-12 base pairs. In any embodiment the second dsDNA molecule can have a variable sequence of 14-18 base pairs. In any embodiment the third dsDNA molecule can have a variable sequence of 24-32 base pairs. In any embodiment the fourth dsDNA molecule can have a variable sequence of 90-110 base pairs. In any embodiment the at least two oligonucleotides can have a variable sequence of 4-6 nucleotides. In any embodiment the anchor strands can have the sequences complementary to the conserved flanking sequences on the at least two oligonucleotides on the 3′ and 5′ ends. In any embodiment the anchor strands can have the sequences that are complementary to the conserved flanking sequences on the at least two oligonucleotides on the 3′ and 5′ ends. In any embodiment the amplification step can be performed by the polymerase chain reaction (PCR). In any embodiment the variable sequence of the anchor strands or of the dsDNA product molecule (e.g. first, second, etc) can be equal to the lengths of the variable sequences on the at least two oligonucleotides. In any embodiment the anchor strand can have a variable sequence present in between the two sequences complementary to the conserved flanking sequences on the at least two oligonucleotides. In any embodiment the anchor strand can have a variable sequence present in between the two sequences complementary to the conserved flanking sequences on the at least two oligonucleotides. In any embodiment the at least two oligonucleotides bound to the anchor strand can abut one another on the anchor strand at their variable sequences. In any embodiment the portion of the variable sequence on the anchor strand that is complementary to the conserved flanking sequence on the at least two oligonucleotides can be 2-6 nucleotides or 14-18 nucleotides or 26-30 nucleotides or 90-110 nucleotides. In any embodiment the at least two oligonucleotides and anchor strand are programmed so that the dsDNA molecule has at least one recognition site for a restriction endonuclease. In any embodiment the restriction endonuclease can be a Type IIS endonuclease. In any embodiment the anchor strand can have 4-6 degenerate nucleotides. In any embodiment the at least one additional dsDNA fragment can be from a parallel synthesis reaction. In any embodiment the 3′ and/or 5′ overhang sequences can have the portion of the variable sequence from the first dsDNA molecule. In any embodiment the step of ligation can occur spontaneously. In any embodiment the at least one additional dsDNA fragment can have a variable sequence at least partially complementary to the variable sequence from the first dsDNA molecule. In any embodiment the methods can further involve a step of ligating the at least two oligonucleotides bound to the anchor strand.

In another aspect the invention provides a composition of at least two oligonucleotides, each comprising a primer binding site on a 3′ or 5′ end (e.g. a universal primer binding site), and a variable sequence on the opposing 5′ or 3′ end, and a conserved flanking sequence in between the primer binding site and the variable sequence. In the composition an anchor strand can have sequences complementary to the conserved flanking sequences on the at least two oligonucleotides, and further have at least one variable sequence, which can be located in between the two sequences complementary to the conserved flanking sequences. At least a portion of the variable sequence on the anchor strand can be complementary to at least a portion of the variable sequences on the at least two oligonucleotides. In one embodiment the anchor strand can have the sequences complementary to the conserved flanking sequences on the at least two oligonucleotides at its 3′ and 5′ ends. The anchor strand can have the variable sequence in between the two sequences complementary to the conserved flanking sequence.

In another aspect the invention provides methods of storing data in a DNA sequence. The methods involve determining a sequence of DNA that encodes a non-genetic message according to a coding scheme that translates the non-genetic message from a reference language into a DNA sequence and vice versa; synthesizing the sequence of DNA that encodes the non-genetic message according to any method disclosed herein; and thereby store data in a DNA sequence.

In another aspect the invention provides methods of synthesizing a DNA sequence encoding a guide RNA. The methods involve determining a sequence of DNA that encodes a guide RNA; synthesizing the sequence of DNA that encodes the guide RNA according to any method disclosed herein.

In another aspect the invention provides an oligonucleotide library comprising 1,536 distinct locations. The library can have 1,024 locations having an oligonucleotide with a unique variable sequence of non-degenerate nucleotides, and an additional 512 distinct locations, each of the 512 locations having an anchor strand with a variable sequence having at least three non-degenerate nucleotides and at least four degenerate nucleotides.

In one embodiment the oligonucleotides have a primer binding site on a 3′ or 5′ end, and a variable sequence on the opposing 5′ or 3′ end, and a conserved flanking sequence in between the primer binding site and the variable sequence. The anchor strands can have conserved flanking sequences complementary to those on the at least two oligonucleotides, and also have at least one variable sequence. At least a portion of the at least one variable sequence on the anchor strand can be complementary to at least a portion of the variable sequences on the at least two oligonucleotides.

In another embodiment the oligonucleotide library has 4,608 distinct locations. 4,096 locations have an oligonucleotide with a unique variable sequence of non-degenerate nucleotides. It can also have an additional 512 distinct locations, each of the 512 locations having an anchor strand with a variable sequence having at least three non-degenerate nucleotides and at least five degenerate nucleotides. The oligonucleotides in the library can be any described herein.

In any embodiment of the libraries some oligonucleotides can contain a 5′ phosphate group (e.g. a deoxyribose 5′ phosphate). In various embodiments at least 25% or at least 30% or about one in three oligos in the library can have a 5′ phosphate. In any embodiment the oligonucleotides containing the 5′ phosphate are not anchor strands. The 5′ phosphate can be present on the 5′ nucleotide. In any embodiment at least 40% or at least 50%, or at least 75% of the oligonucleotides in the library that are not anchor stands can contain a 5′ phosphate. In one embodiment all the “O2” oligonucleotides have a 5′ phosphate. The 5′ phosphate on one of two oligos to be joined can be helpful for the action of a ligase. Locations in the library containing anchor strands can contain anchor strand oligonucleotides having every possible sequence of the variable sequence, i.e. there can be a plurality of oligonucleotide sequences at the location. The variable sequence of the anchor strand can have five or six nucleotides, or can be otherwise as the anchor strands described herein. At other locations in the library (e.g. containing non-anchor strand oligonucleotides) there can be present an oligonucleotide of unique sequence, i.e. a single sequence at the location.

### DETAILED DESCRIPTION OF THE DRAWINGS

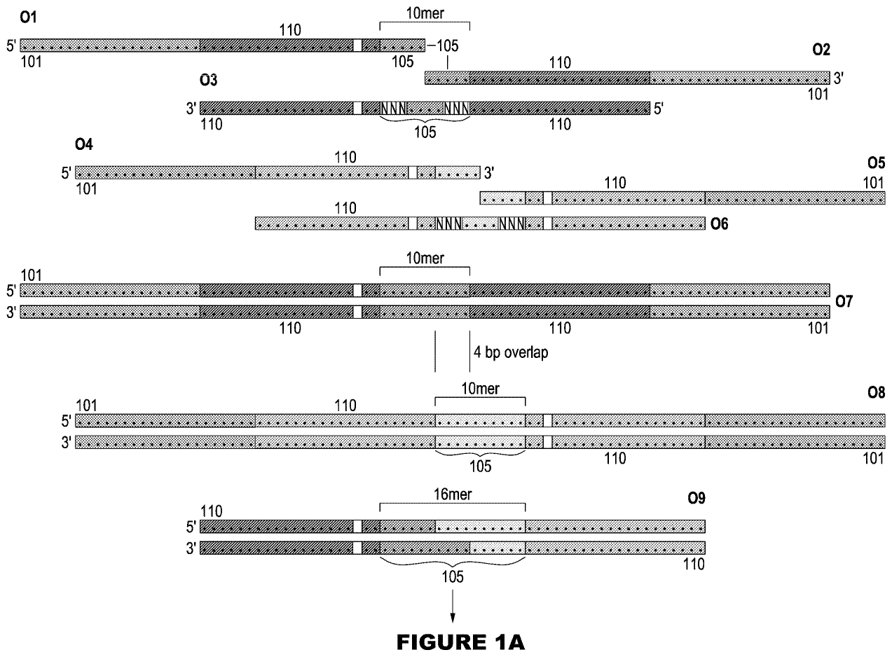

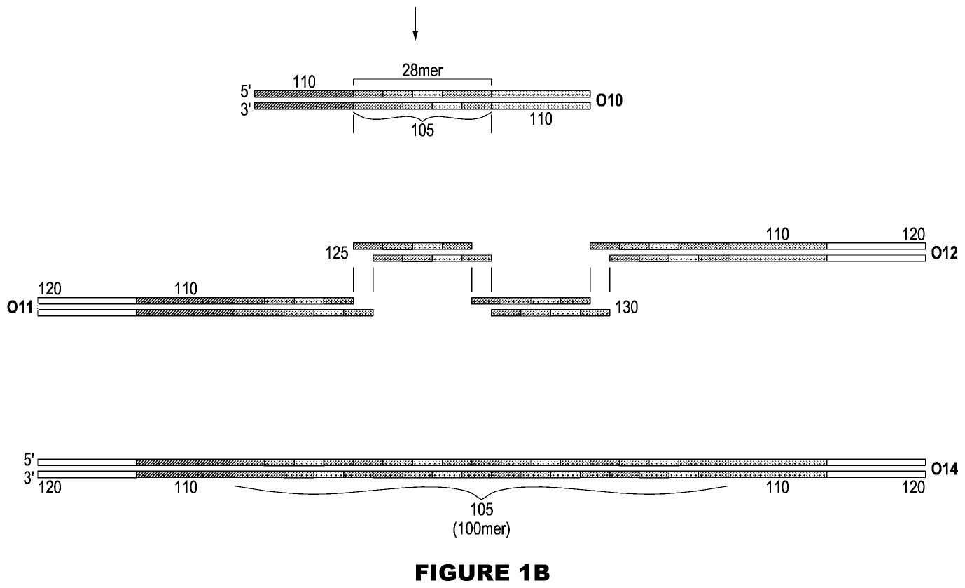

FIGS. 1A-1B. FIG. 1A provides a schematic illustration of the synthesis of a DNA molecule of desired sequence according to one embodiment of the invention. FIG. 1B provides further reactions from the product of a schematic illustration of the synthesis of a DNA molecule of desired sequence. Degenerate nucleotides are labeled as “N”.

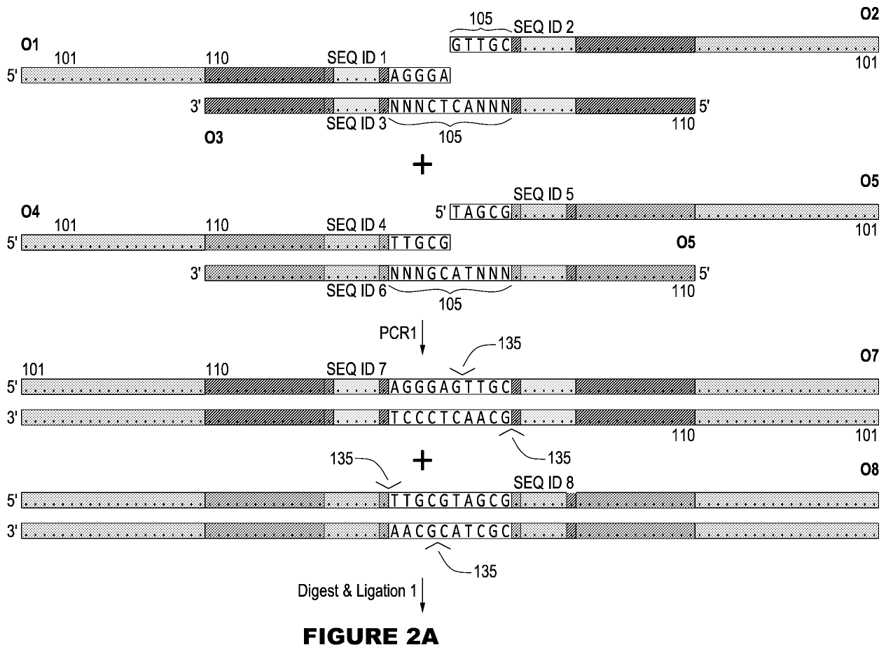

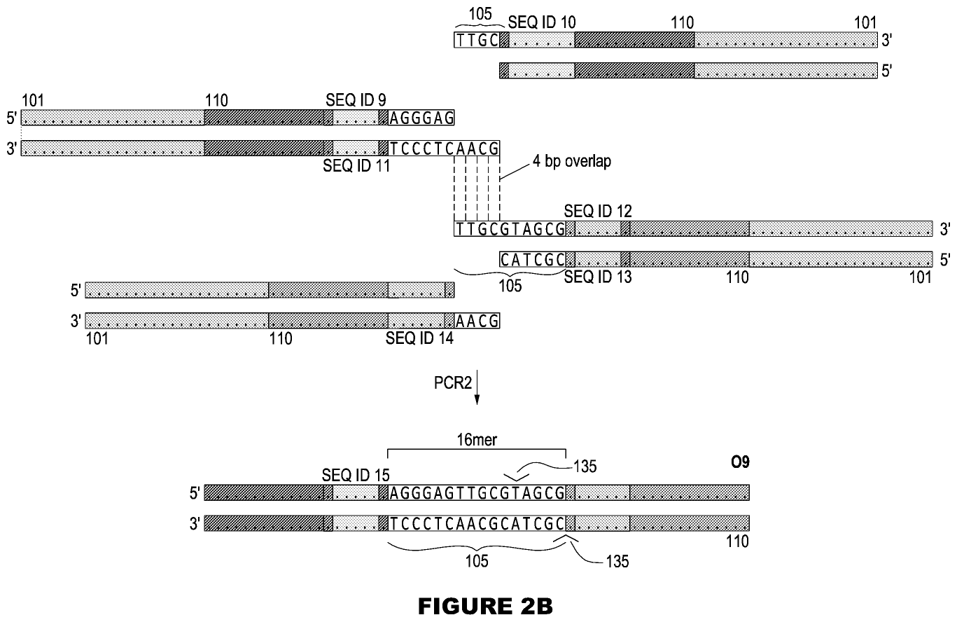

FIGS. 2A-2B. FIG. 2A provides a schematic illustration containing details of an embodiment of the DNA synthesis reaction of the invention. FIG. 2B shows an additional part of the reaction. The illustrations show complementary 3′ and 5′ overhang sequences between dsDNA molecules and fragments. Degenerate nucleotides are labeled as “N”.

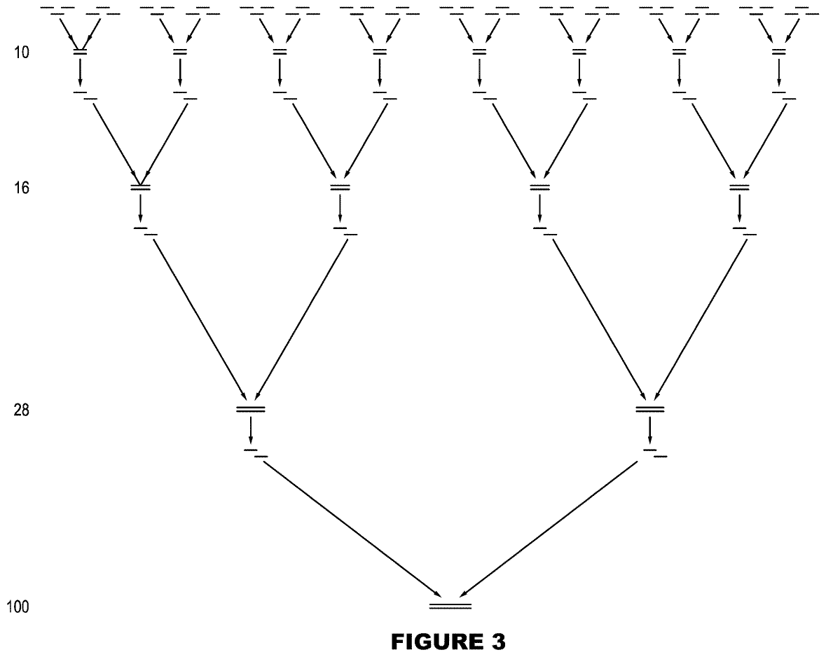

FIG. 3 provides a schematic illustration of an embodiment of an overall scheme for hierarchal assembly using the methods of the invention. By synthesizing dsDNA molecules with overlapping variable sequences hierarchal assembly can be leveraged. Assembly can also include the addition of dsDNA fragments with a 5′ and 3′ overhang, which can be added to the final assembly (or any assembly step). Lengths of variable sequences are for illustration only.

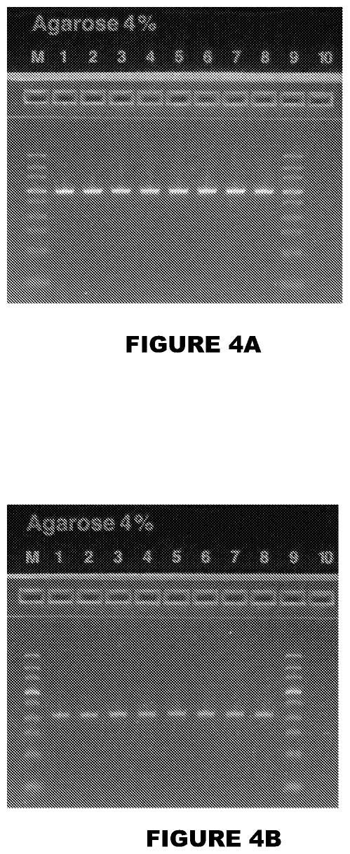

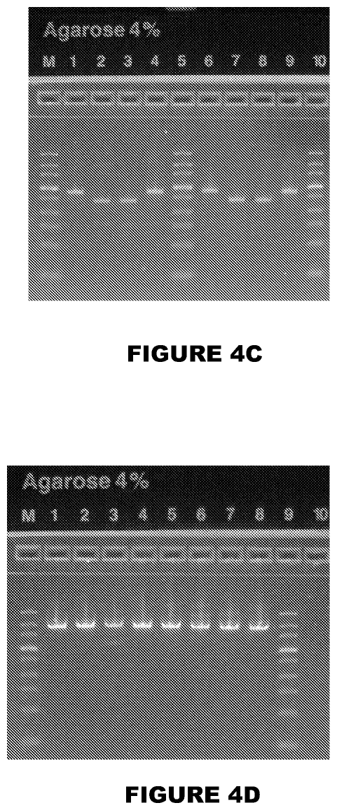

FIGS. 4A-4D. FIG. 4A provides a gel image of PCR1 products after the first ligation step (L0) where three oligos from the library are combined, ligated and PCR amplified using a single universal primer pair. Contained within each of the 98 bp PCR products is 10 bp of synthetic DNA (variable sequence) that is leveraged for downstream assembly. FIG. 4B provides a gel image showing PCR2 products after the first digest and ligation step (DL1) on PCR1 products. These PCR2 products resulted from combining two PCR1 products, removal of one flanking sequence on each product (by enzymatic digestion) and then ligation of the products. Although the PCR2 product total length is shorter (56 bp) than the PCR1 product in FIG. 1, the variable sequence of the dsDNA has increased to 16 bp, representing an increasing variable sequence length as the workflow progressed. FIG. 4C provides a gel image showing PCR3 products after the second digestion and ligation step (DL2). These PCR3 products resulted from combining two PCR2 products, digesting away one flanking sequence on each product followed by ligation. Note that the 1st and 4th PCR product contain a 5′ phosphorothiolate capped termini, which aids in reducing downstream mis-ligation events, plus these also provide a universal priming sequence for the subsequent PCR4 amplification. The variable sequence of the PCR3 dsDNA molecule is 28 bp in length in all the sequences on this gel. FIG. 4D provides a gel image showing PCR4 products after the third digestion and ligation step (DL3). These PCR4 products result from combining four PCR3 products, digesting away one or two flanking sequences on each product and then ligating them together. The variable sequence of the PCR4 dsDNA is 100 bp in length in all the sequences on this gel, and they have a 40 bp flanking sequence on both sides, which can be digested away by using BsmBI to enable further assembly into even larger pieces of DNA.

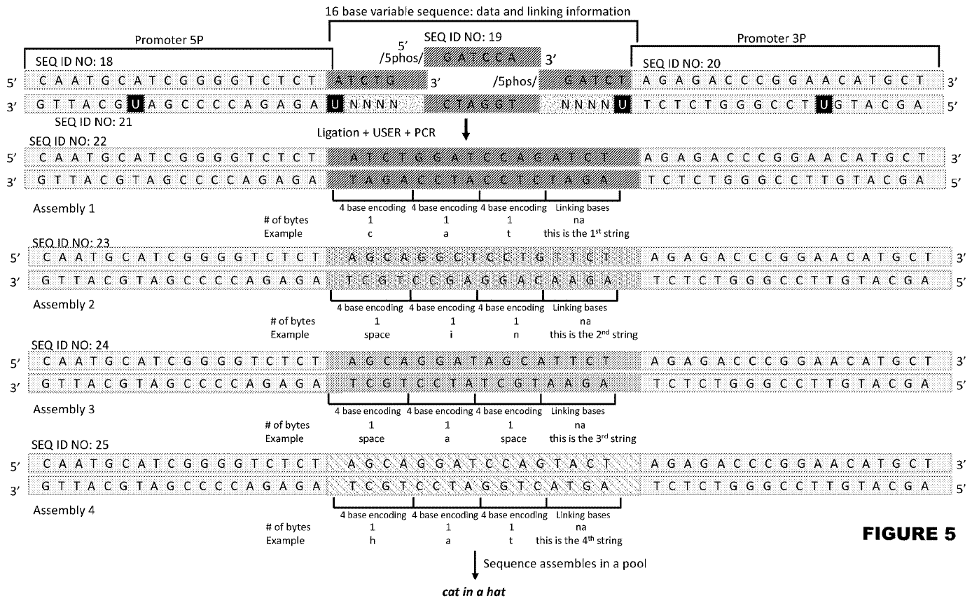

FIG. 5 provides a schematic illustration of an embodiment of the invention for storing digital information in DNA A 16 bp product DNA molecule is produced encoding four bytes of information. The examples shows how a non-genetic message (here “cat in a hat”) can be encoded into DNA using the methods of the invention.

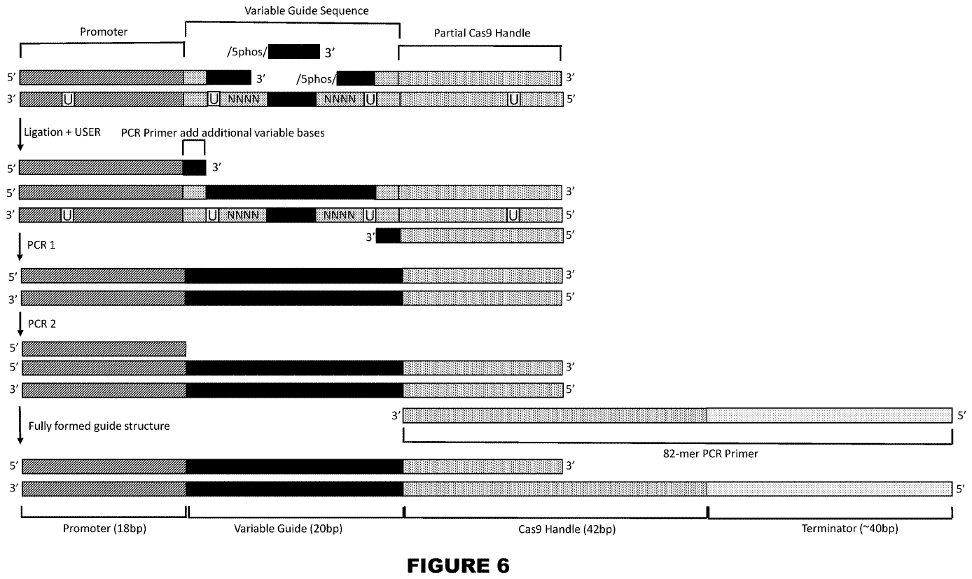

FIG. 6 is a schematic illustration of an embodiment of the invention applied to synthesizing a 120 bp product DNA, which is an initial guide structure having the transcriptional elements of a promoter, a guide RNA, a Cas9 handle, and a terminator. In this embodiment the first cycle of PCR utilizes two primers having two variable bases on their 3′ ends. This converts the otherwise 16 bp product DNA molecule into a 20 bp product. Later step(s) of PCR incorporate transcriptional elements.

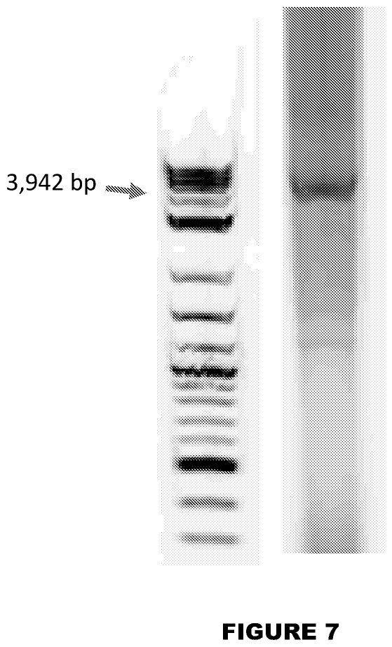

FIG. 7 is a diagram of a gel image showing the fully synthetic, assembled 3,942 bp spike gene. The full length was confirmed by cloning and DNA sequencing and determined to have an error rate of approximately 1 error per 5,400 bp, prior to applying an enzymatic error correction step.

### DETAILED DESCRIPTION OF THE INVENTION

The invention provides methods of assembling DNA molecules of any sequence with high fidelity using a universal library of oligonucleotides. The methods involve the use of an oligonucleotide library having DNA molecule members such that all possible DNA sequences can be assembled from the library using the methods. In one embodiment the library of oligonucleotides has less than 10,000 members. Many efforts have been made towards achieving methods of assembling any possible DNA sequence from a library having a limited number of members. The present inventors discovered that any possible DNA sequence can be conveniently assembled using the materials and methods disclosed herein. The invention therefore enables creation of a library of less than 10,000 oligonucleotides, from which all possible oligonucleotide sequences can be assembled. The library of less than 10,000 oligonucleotides can be conveniently provided on a small device (e.g. a DNA chip), and devices and instrumentation provided to selectively assembly any DNA sequence using only the members of the oligonucleotide library.

### Oligo Library

In various embodiments the oligonucleotide members in the library can be DNA of various lengths. In various embodiments the library of oligonucleotides can have less than 20,000 members or less than 15,000 or less than 12,000 members, or less than 10,000 members, or less than 9,000 members, or less than 8,000 members, or less than 7,000 members, or less than 6,000 members.

In various embodiments the library can contain at least 2,000 members, or at least 3,000 members, or at least 5,000 members, but nevertheless also contain less than 20,000 members or less than 15,000 or less than 12,000 members, or less than 10,000 members, or less than 9,000 members, or less than 7,000 members, or less than 6,000 members, in all possible combinations and sub-combinations. The methods are able to synthesize all possible polynucleotide sequences using the oligonucleotide members in the library. In various embodiments the invention permits the assembly of over 4 billion (for a 16mer) and up to over 1 trillion (for a 20mer) polynucleotides of distinct sequence (e.g. variable sequence) beginning only with the oligonucleotides in the library. In various embodiments each oligonucleotide in the library can be used from 100 to 10,000 times in the synthesis of product DNA molecules.

The product DNA molecule assembled can be of any size, for example it can be more than 100 bp, or more than 250 bp, or more than 500 bp, or more than 750 bp, or more than 1 kbp, or more than 1.5 kbp, or more than 2 kbp, or more than 5 kbp or more than 10 kbp, or 100 bp-500 bp, or 80-500 bp, or 80-750 bp, or 80-1000 bp, or 1-4 kbp, or 1-5 kbp, or 2-10 kbp or 5-15 kbp or 5-20 kbp, or up to 10,000 bp, or up to 7,000 bp, or up to 5000 bp, or up to 4000 bp, or less than 1 kbp, or less than 750 bp, or less than 500 bp, or less than 250 bp, or less than 500 kbp or less than 1 Mbp or less than 5 Mbp or less than 10 Mbp or less than 12 Mbp, or less than 13 Mbp, or less than 14 Mbp, or less than 15 Mbp or 1-10 Mbp, or 1-12 Mbp, or 1-15 Mpb, whether counting or not counting conserved flanking sequences and primer binding sequences. These lengths can exist and are disclosed in all combinations and sub-combinations of lengths above. The terms “oligo” and “oligonucleotide” are used interchangeably herein and indicates a polymer of nucleotides of generally shorter length. “Polynucleotide” is a general term denoting a polymer of nucleotides of any length. The library can consist of any of the oligonucleotides described herein.

In other embodiments the methods can also be used with even smaller libraries to assemble a significant number of sequences that may be desired, for example to assemble a more limited and directed number of sequences in a defined category where such sequences are needed. Examples of a defined category can include a set of genes related to a specific biological function, or genes from a particular organism. In any embodiment the product DNA molecule synthesized in the method can be synthesized entirely from and only using oligonucleotides from the oligonucleotide library. A “universal library” is a library of polynucleotide molecules from which any possible DNA sequence can be assembled. At a broad level a universal library can contain polynucleotides that can be assembled into any possible DNA sequence. However, within particular defined categories of DNA sequences smaller universal libraries (or defined category libraries) can be used containing sequences of interest, for example a library of DNA sequences for RNA metabolism, or for genes or sequences related to transcription, or for regulation, RNA metabolism, translation, protein folding, protein export, RNA (rRNA, tRNA, small RNAs), ribosome biogenesis, rRNA modification, DNA replication, DNA repair, DNA topology, DNA metabolism, chromosome segregation, cell division, and tRNA modification. Thus, in some embodiments any of these, and any other category, can be considered a defined category library of sequences of interest for a more specific purpose. Definitions of DNA sequences to be included in a defined category library or library of sequences of interest may be subject to some discretion of the user depending on the needs of the application. Thus, the methods disclosed herein can assemble all possible sequences of interest, which is a sub-set of literally all possible sequences.

### Oligonucleotides

In one embodiment the at least two oligonucleotides can be DNA of any convenient length. For example, the at least two oligonucleotides can be greater than 12 nucleotides in length or, without limitation, about 20-65 nucleotides, or 20-35 nucleotides, or 35-55 nucleotides, or 25-65 nucleotides, or 30-60 nucleotide, or 40-50 nucleotides, or 40-60 nucleotides, or about 42-48 nucleotides, or about 44 or about 45 nucleotides. Anchor strands used in the method can be from 20-60 nucleotides, or from 20-70 nucleotides, or from 30-60 nucleotides or from 30-70 nucleotides, or from 35 to 65 nucleotides, or from 40-60 nucleotides or from 40-50 nucleotides. In one embodiment the at least two oligonucleotides are from 40-50 nucleotides and the anchor strand is from 35-45 or from 45-55 nucleotides. Primer binding sites can be added to or included in these oligonucleotide lengths. Oligonucleotides can be present in any combination or sub-combination of the lengths provided herein. In any embodiment the oligonucleotides can have only nucleotides having no non-standard bases. In any embodiment the oligonucleotides can have only nucleotides having standard bases, i.e. all nucleotides in the oligonucleotide have a standard base that is either A (adenine), T (thymine), C (cytosine), or G (guanine). In other embodiments any of the oligonucleotides can contain one or more non-standard bases. The oligonucleotides and/or anchor strands can have sequences for binding a primer, which can be used in PCR or another DNA amplification procedure. In sizing nucleotides for the library the person of ordinary skill with resort to this disclosure will realize optimal sizes of oligonucleotides to use in the methods by considering the ability of oligo lengths to anneal to other oligos. Any of the oligonucleotides can be programmed and synthesized to have a recognition site for a restriction endonuclease in a resulting dsDNA molecule. The restriction enzyme can be one that recognizes asymmetric DNA sequences and cleaves a number of nucleotides outside of their recognition sequence (e.g. within 1-5 or 1-10 or 1-20 nucleotides), e.g. Type IIS restriction enzymes. Any of the oligonucleotides described herein can be members of the oligo library, including all combinations and sub-combinations of described oligonucleotides.

The methods of the invention synthesize a product DNA molecule having a “desired sequence,” which can be a pre-determined sequence, i.e. one decided by the user prior to initiating the method. The product DNA molecule of desired sequence can be any molecule produced by the method including but not limited to the first dsDNA molecule, the second dsDNA molecule, the third dsDNA molecule the fourth dsDNA molecule, and the additional dsDNA molecule. The dsDNA fragments or additional dsDNA fragments can be derived from a restriction enzyme digestion of any product dsDNA molecule. In various embodiments the DNA molecule of desired sequence can be at least 16 bp, or at least 20 bp, or at least 30 bp, or at least 40 bp, or at least 60 bp, or at least 80 bp or at least 90 bp, or at least 100 bp, or at least 175 bp, or at least 200 bp, counting or not counting conserved flanking sequences or primer binding sites. The oligonucleotides (and/or anchor strand) utilized in the methods can contain a variable sequence, which can correspond a portion of the variable sequence of the product dsDNA molecule. Thus, in any embodiment the product dsDNA molecule can be considered with or without primer binding sites added to the 3′ and 5′ ends. In any embodiment the variable sequence or sub-sequence of the oligonucleotides and/or anchor strand can be at least 4 bp, or at least 5 bp, or at least 8 bp, or at least 16 bp or at least 28 bp or at least 100 bp. The length of variable sequence of the product DNA molecule can depend on the step in the method and can be provided through the combination of oligonucleotides and dsDNA fragments. In various embodiments the length of the variable sequence can be at least 8%, or at least 10%, or at least 15%, or at least 20%, or at least 25% of the oligonucleotide or anchor strand length. In other embodiments the length of the variable sequence of the dsDNA molecule can be at least 8%, or at least 10%, or at least 25%, or at least 50%, or at least 60%, or at least 75% of the product dsDNA molecule.

### Methods

FIG. 1A depicts a method of synthesizing a product dsDNA molecule according to a method of the invention. O1 and O2 are the at least two oligonucleotides, and O3 is the anchor strand. In this embodiment O1-O2 each have a primer binding site 101 (which can be a universal primer binding site) on the 5′ or 3′ end, and a variable sequence 105 on the opposing 3′ or 5′ end. O1-O2 also each have a conserved flanking sequence 110, in this embodiment depicted in between the universal primer binding site 101 and the variable sequence 105. In this embodiment O1 and O2 are 40-50 or about 45 nucleotides in total length. The “conserved flanking sequence” (CFS) serves to assist the oligos in annealing to target oligos having complementary CFSs, and can also have primer binding sites (e.g. for use later in the methods). In various embodiments the CFSs can be a sequence of at least 8 nucleotides, or at least 12 nucleotides, or at least 15 nucleotides, or 15-25 nucleotides, 18-22 nucleotides, or 8-15 nucleotides, or 10-18 nucleotides, or 12-25 nucleotides, or 12-30 nucleotides, or 15-20 or 15-30 nucleotides, 18-22 nucleotides or 18-30 nucleotides or 18-60 nucleotides, but any convenient length can be used that is able to aid in annealing and provide a primer binding site. In a particular embodiment the CFS is 18-22 or about 20 nucleotides. In various embodiments CFSs on an anchor strand can be complementary to CFSs on the at least two oligonucleotides, with which they bind (e.g. oligos in a binding set). In various embodiments the conserved flanking sequence can be present on each molecule in a binding set. The conserved flanking sequence can be located on the at least two oligonucleotides in between the primer binding site and the variable sequence. In any embodiment conserved flanking sequences can include a 5′ cap to discourage degradation of the dsDNA molecule, and/or primer binding sequences to aid amplification.

In this embodiment the anchor strand O3 has, at the 3′ and 5′ ends, conserved flanking sequences 110 complementary to the conserved flanking sequences on the at least two oligonucleotides (or to at least a portion of the conserved flanking sequences on the at least two oligonucleotides sufficient to anneal the oligos). In this embodiment O3 is about 50 nucleotides in length, with the CFSs being about 20 nucleotides each. O3 also has at least one variable sequence 105, in this embodiment (of an anchor strand) situated in between the two conserved flanking sequences 110 and depicted as being about 10 nucleotides in length. In other embodiments the variable sequence can be moved to another location on the oligos, as long as sufficient space is left for a CFS able to facilitate annealing and/or provide a primer binding site (if utilized). In any embodiment at least 10 or at least 15 or at least 18 nucleotides of a conserved flanking sequence can be present on both sides of the variable sequence of the anchor strand. In this embodiment the variable sequence 105 on the anchor strand O3 comprises degenerate nucleotides N, here six degenerate nucleotides as depicted in FIG. 1A. At least a portion of the at least one variable sequence 105 on the anchor strand is complementary to at least a portion of the variable sequences 105 on the at least two oligonucleotides, and in one embodiment can be complementary across the whole variable sequence. One or more of the at least two oligonucleotides can further be synthesized to have a recognition site for a restriction endonuclease when assembled into a dsDNA molecule; the anchor strand can also be synthesized to contain a recognition site for a restriction endonuclease so that, when bound to the at least two oligonucleotides the recognition sites are present and active. In one embodiment the restriction endonuclease can be a Type IIS restriction endonuclease. The recognition site on the at least two oligonucleotides and anchor strand can be programmed to lie outside of the variable sequence on the assembled molecule, but the restriction endonuclease can cut inside of the variable sequence. In one embodiment the recognition sites are comprised within the conserved flanking sequence; and in one embodiment the restriction endonuclease cleaves within the variable sequence of a dsDNA molecule. In any embodiment, any two nucleotide sequences that are complementary or overlapping can have at least 90% sequence identity, or at least 95% sequence identity, or at least 98% sequence identity, or 100% sequence identity in the nucleotide sequences. The product DNA molecule can be optionally assembled having conserved flanking sequences, useful for continuing procedures (e.g. PCR or other DNA amplification).

The method involves steps of annealing the at least two oligonucleotides O1-O2 to an anchor strand O3 so that the at least two oligonucleotides bound to the anchor strand abut one another on the anchor strand. In one embodiment the at least two oligonucleotides can abut at their variable sequences. In one embodiment the variable sequences 105 of the at least two oligonucleotides are annealed to the variable sequence of the anchor strand 105. It is noted in the embodiment illustrated that each of O1-O2 have a variable sequence of 5 nucleotides and O3 has a variable sequence of ten nucleotides. Upon annealing the respective variable sequences anneal and form base pairs. In one embodiment the variable sequences form one continuous sequence after ligation. In the invention “binding” or “annealing” are used interchangeably with respect to polynucleotides and refer to formation of a double-stranded DNA molecule by standard Watson-Crick base pairing. In various embodiments annealing can occur when at least 25% or at least 50% or at least 75% or at least 90% of the nucleotides are base paired to the complementary oligonucleotide(s). Two oligonucleotides “abut” one another when a first oligonucleotide contains a nucleotide that is present adjacent to a nucleotide on a second oligonucleotide when both oligonucleotides are bound to the same (third) complementary oligonucleotide (e.g. an anchor strand) by Watson-Crick base pairing, at least at their variable sequences, i.e. the nucleotides are based paired to nucleotides that are adjacent to one another on the complementary (e.g. anchor) strand. In any embodiment the anchor strand can be completely bound to the first and second oligonucleotides. FIGS. 1 and 2 illustrate this concept. Thus, when abutting, each nucleotide of the anchor strand can be annealed to a nucleotide of one of the at least two oligonucleotides. After an amplification reaction each nucleotide of the at least two oligonucleotides can be annealed to a nucleotide on the anchor strand. In any embodiment the anchor strand can be annealed to two, and not more than two, oligonucleotides.

The methods also involve a step of ligating the at least two oligonucleotides annealed to the anchor strand to produce a (“first”) dsDNA molecule. The step of ligation or “ligating” can mean contacting the annealed dsDNA fragments or dsDNA molecules with a ligase, or allowing ligation to occur spontaneously. A ligase is an enzyme that catalyzes the joining of two polynucleotide molecules by forming a new chemical bond. In one embodiment the ligase can ligate adjacent (or abutting) polynucleotides bound to the same complementary polynucleotide strand. In any of the methods any DNA ligase can be used, for example T4 DNA ligase and E. coli DNA ligase are just two examples, but another DNA ligase can also be used.

The methods can involve a step of performing an amplification step to produce a product dsDNA molecule. In any step of any of the methods the amplification step can involve, for example, PCR, isothermal amplification, rolling circle amplification, loop-mediated isothermal amplification, or another DNA amplification method on the dsDNA molecules (e.g. O1-O3 and O4-O6 when present) to produce a first dsDNA molecule O7 (and/or O8). In the embodiment of FIG. 1 the variable sequences of O7 and O8 are 10mers as an example, but persons of ordinary skill with resort to this disclosure will realize that any appropriate length of variable sequence can be used in the methods, depending on the step in the methods. For example variable sequences of 6-20 or 6-12 or 6-14 or 8-12 or 10-16 or 15-25 or 20-30 or 30-50 or 40-100 or 60-120 nucleotides or other numbers of nucleotides can be used on anchor strands in any embodiment and, optionally, can correspond in length to the sum of the variable sequences on the at least two oligonucleotides or dsDNA fragments from which a dsDNA molecule is synthesized (minus overlapping nucleotides). In any embodiment the at least two oligonucleotides can have variable sequences of different lengths. For example, one of the two oligonucleotides can have a variable sequence of 4 nucleotides and the second oligo a variable sequence of 6 nucleotides or other combinations to form a first dsDNA molecule. In the embodiment depicted in FIG. 1 the first dsDNA molecule (e.g. O7 or O8) is synthesized with universal primer binding sites 101 at the 3′ and 5′ ends, a conserved flanking sequence 110 inside each of the 3′ and 5′ ends, and a variable sequence 105 inside the conserved flanking sequences 110. This arrangement can be utilized in any embodiment. With respect to dsDNA molecules “inside” refers to a feature present further towards the center of the DNA molecule (and further away from the 5′ or 3′ ends) than a reference feature.

In some embodiments a plurality of product DNA molecules can be “multiplexed,” i.e. synthesized in the same reaction pool. In other embodiments where desired, DNA molecules can be synthesized individually (e.g. “in parallel”) in their own reaction pools (and combined subsequently). Reactions can be multiplexed with two or more binding sets of the at least two oligonucleotides and at least one anchor strand. As with any of the methods, the method depicted in FIG. 1 can be performed as an individual synthesis of one dsDNA molecule, or as a multiplexed synthesis of at least two dsDNA molecules in the same pool. Synthesized DNA molecules can later (optionally) be joined by the methods disclosed herein, e.g. as illustrated in FIGS. 1-2. When multiplexing is utilized, more than one DNA molecule or more than two DNA molecules are synthesized in a simultaneous reaction in the same pool. In FIG. 2A multiplexing is depicted as paired oligos O4-O5 and paired anchor strand O6 forming a separate paired dsDNA molecule O8, as a pair to the first dsDNA molecule O7. But a paired dsDNA molecule (e.g. O8) can be synthesized in a parallel and separate reaction pool, and the first and paired dsDNA molecules (or fragments therefrom) combined in a subsequent step. In any embodiment the dsDNA molecules (or dsDNA fragments) are “paired” when they have an overlapping sequence at the variable sequence. In this embodiment there is depicted variable sequences of 10 bp in the first and paired dsDNA molecule and an overlap of 4 bp in the variable sequence between the dsDNA molecules (e.g. as in FIG. 2B). But in any embodiment of the methods the overlap can be at least 1 bp, or at least 2 bp or at least 3 bp or at least 4 bp, or at least 5 bp or at least 6 bp or at least 8 bp or at least 12 bp or more than 12 bp. Any two dsDNA molecules (or dsDNA fragment), whether first dsDNA molecule, second, third, fourth dsDNA molecule, etc., or additional dsDNA molecule can be dsDNA molecules (or dsDNA fragments) that are paired.

dsDNA fragments can be produced (e.g. by restriction enzyme action on a dsDNA molecule or, in any embodiment, separately synthesized) to produce paired dsDNA fragments that have overhanging 3′ and/or 5′ sequences, which overhangs can be at their variable sequences and can at least partially overlap. Such dsDNA fragments can therefore be annealed at the 3′ and/or 5′ overhangs to form a larger dsDNA molecule. “Overlapping” (or complementary) sequences are those that comprise a complementary sequence for a series of nucleotides sufficient to be annealed under standard reaction conditions by Watson-Crick base pairing. In any embodiment the methods can utilize dsDNA fragments or polynucleotides that overlap by 1, 2, 3, 4, 5, 6, 7, or 8 nucleotides (which can be consecutive nucleotides), or by at least 1 or at least 2 or at least 3 or at least 4 or at least 5 or at least 6 or at least 7 or at least 8, or at least 12, or at least 15 nucleotides, any of which can be consecutive nucleotides. In any embodiment the overlap can be at their variable sequences. “Overhangs” or “overhanging” sequences refers to 3′ or 5′ single-stranded DNA sequences that extend from a double-stranded DNA sequence. In various embodiments the overhangs can be at least 2 or at least 3 or at least 4 nucleotides, or at least 6 or at least 8 or at least 10 nucleotides. At least two paired oligonucleotides can anneal or bind to their corresponding paired anchor strand. The “paired anchor strand” is simply an anchor strand having sequences complementary to the at least two paired oligonucleotides so that they can be annealed sufficient to function in the method.

The methods can involve further steps towards synthesizing a larger product DNA molecule. The methods can involve a step of contacting a first dsDNA molecule and a paired dsDNA molecule (e.g. O7-O8) with a restriction enzyme to produce first and paired dsDNA fragments that have 3′ and/or 5′ complementary overhang sequences and a portion of the variable sequence from the first and paired dsDNA molecules, respectively (illustrated in one embodiment in FIG. 2B). In any embodiment the first and paired dsDNA molecules can have variable sequences that are complementary and overlapping, and can have conserved flanking sequences containing a recognition site for a restriction endonuclease (e.g. a Type IIS restriction endonuclease). The restriction enzyme can cleave within the variable sequence of the first dsDNA molecule (and paired dsDNA molecule when present) to produce complementary overhanging 3′ and/or 5′ sequences on the first dsDNA fragment and paired dsDNA fragment.

The methods can involve a step of providing at least one additional dsDNA fragment that has a 3′ and/or 5′ overhang sequence complementary to an overhang sequence of at least one other dsDNA fragment in the synthesis method (e.g. the first or paired dsDNA fragment). The overhang sequence of the additional dsDNA fragment can contain at least a portion of the variable sequence (e.g. as depicted in FIG. 2). The overhang sequences can comprise at least a portion of the variable sequence of the first dsDNA molecule (and paired dsDNA molecule when present), and therefore the fragments can have overlapping and complementary sequences at the variable sequence. The at least one additional dsDNA fragment can be from a restriction endonuclease reaction on the dsDNA molecule(s) or its paired dsDNA (e.g. O7 and O8), or can be a DNA fragment produced in another, parallel reaction, and can also be separately synthesized. Each of these dsDNA fragments have a complementary 3′ or 5′ overhang sequence to at least one other dsDNA fragment in the method. However, in other embodiments a plurality of additional dsDNA fragments can be imported and assembled into the product dsDNA molecule at the same time. Some dsDNA fragments can have both a 3′ and a 5′ overhang sequence complementary to two other dsDNA fragments in the reaction (e.g. as in FIG. 1B), and can therefore be inserted in between two fragments to lengthen the product DNA molecule—these dsDNA fragments therefore will have 3′ and 5′ overhangs, each complementary to one other 3′ or 5′ overhang of a dsDNA fragment in the synthesis.

The additional dsDNA fragment(s) can be used in any embodiment. “Additional dsDNA fragment” (or additional dsDNA molecule) is a general term, not necessarily specific to any particular step in the methods. Such additional dsDNA fragments can be annealed to another dsDNA fragment having a complementary sequence at any step in the methods, e.g. at 3′ and/or 5′ overhangs. The additional dsDNA fragment can have a sequence at least partially complementary to the 3′ and/or 5′ overhang on at least one other dsDNA fragment in the method, which can be the first dsDNA fragment, or second dsDNA fragment, or third dsDNA fragment, or fourth dsDNA fragment, or another additional dsDNA fragment. When a dsDNA molecule is cut with a restriction endonuclease it can leave the 3′ and 5′ overhangs. Thus, if two dsDNA molecules are cut with a restriction endonuclease the resulting two dsDNA fragments can be annealed and joined by their complementary 3′ and 5′ overhangs (e.g. as illustrated in FIG. 2B). In various embodiments the complementarity or overlap can be over at least 5 or at least 6 or at least 8 or at least 10 or at least 12 or at least 15 nucleotides, which can be consecutive nucleotides. In any embodiment the overhang of the additional dsDNA fragment, or any dsDNA fragment, can be in the variable sequence. In any embodiment additional dsDNA fragments can be derived from additional dsDNA molecules, e.g. by cutting with a restriction endonuclease to produce additional dsDNA fragments. The additional dsDNA fragments can therefore have at the 3′ and/or 5′ overhangs the next series of nucleotides to be synthesized into the final product dsDNA molecule to form the dsDNA molecule of desired (pre-determined sequence). In any embodiment any additional dsDNA molecule can have the same structures as the first dsDNA molecule, or second dsDNA molecule, etc. (but varying at the variable sequence). And any additional dsDNA fragment can have the same structures as the first dsDNA fragment, second dsDNA fragment, etc. For example, in any embodiment the additional dsDNA molecule can have a primer binding site on a 3′ and/or 5′ end, a variable sequence, and conserved flanking sequences on either side of the variable sequence the same as, for example, O7 or O8. An additional dsDNA fragment can be (but is not necessarily) derived from restriction enzyme digestion of an additional dsDNA molecule.

The methods can involve a step of annealing a first dsDNA fragment with at least one paired or additional dsDNA fragment by their complementary overhang sequences, which overlap can be at their variable sequences. The methods also can involve performing a step of ligation to produce a second dsDNA molecule (e.g. O9, FIG. 1A) (here depicted having a 16mer variable sequence) having a conserved flanking sequence (CFS) 110 inside each of the 3′ and 5′ ends, and a variable sequence 105 inside the 3′ and 5′ conserved flanking sequences that is longer than the variable sequence on the first dsDNA molecule.

The methods therefore can further involve a step of contacting at least one second dsDNA molecule with a restriction enzyme to produce a plurality of second dsDNA fragments comprising 3′ and/or 5′ overhang sequences. At least two of the plurality can have a conserved flanking sequence inside each of the 3′ and/or 5′ overhangs. The method can further involve a step of annealing at least one of the second dsDNA fragments to one or more paired or additional dsDNA fragments having a complementary 3′ or 5′ overhang sequence (which can be at the variable sequence), and performing a step of ligation to produce at least one third dsDNA molecule having a conserved flanking sequence on the 3′ and 5′ ends (or, optionally, inside of primer binding sites on the 3′ and 5′ ends), and a variable sequence inside the conserved flanking sequences that is longer than the variable sequence of the second dsDNA molecule. In the embodiment depicted in FIGS. 1 and 2 the at least one third dsDNA molecule has a variable sequence of 28 bp and a 4 bp overlap with an at least one paired third dsDNA molecule.

The methods can further involve a step of reacting the at least one third dsDNA molecule with a restriction enzyme to produce at least one third dsDNA fragment e.g. O11 having 3′ and/or 5′ overhang sequences, optionally annealing the at least one third dsDNA fragment to one or more paired or additional dsDNA fragments e.g. O12 having a complementary 5′ or 3′ overhang sequence, and performing a step of ligation to produce a fourth dsDNA molecule. At least two of the dsDNA fragments in the mixture can have a conserved flanking sequence inside the variable sequence, and a variable sequence at the 3′ or 5′ overhang. The fourth dsDNA molecule can therefore have conserved flanking sequences inside the 3′ and 5′ ends, an optional primer or universal primer binding sequence 120, and a variable sequence (optionally between the CFSs) that is longer than the variable sequence of the third dsDNA molecule. In the embodiment depicted in FIGS. 1 and 2 the at least one fourth dsDNA molecule has a variable sequence of 100 bp O14. As in the other steps one or more additional dsDNA fragments can be included into the reaction to further lengthen the variable sequence of the product dsDNA molecule. The at least one additional dsDNA fragment can be derived from parallel (or multiplexed) reactions. As depicted in FIG. 1B as fragments 125 and 130 one or more of the dsDNA fragments can have overhangs at both the 3′ and 5′ ends with a sequence complementary to the overhang sequences of two other dsDNA fragments in the reaction. These additional dsDNA fragments can be produced by including two restriction recognition sites on the dsDNA molecule (optionally within the CFSs) contacted with the restriction endonuclease, which can then cleave the dsDNA molecule into at least three fragments. The dsDNA fragments can therefore be joined in annealing and ligation reactions to form a longer product dsDNA molecule. In any of the embodiments or steps of the methods two or more dsDNA fragments can be included in the reaction, and the dsDNA fragments can have a 3′ and 5′ overhang sequence and not have a CFS, i.e. these dsDNA fragments having both a 5′ and 5′ overhang sequence can be all variable sequence.

The methods offer great versatility in synthesizing a product dsDNA molecule of desired sequence. In any embodiment the dsDNA molecule can be synthesized from multiple dsDNA fragments, e.g. two, or three, or four, or five, or six, or more than six fragments. The multiple fragments can each comprise a portion of the product dsDNA molecule to be synthesized. In any embodiment, a step in synthesis or the final step in synthesis can include at least one dsDNA fragment in the annealing reaction that includes at least a portion of the desired sequence so that the desired sequence is present on the product dsDNA molecule. For example, in any embodiment a 5′ cap and/or primer binding site 120 can be added to the 3′ and/or 5′ ends of the product dsDNA molecule. In embodiments where multiple fragments are used in synthesis, the first of the fragments on the 5′ end and the last of the fragments on the 3′ end of the assembled molecule can have a 5′ cap. The 5′ cap can assist in preventing degradation of the ends of the DNA molecule, and the priming sequence is convenient for amplification when desired. In various embodiments the 5′ cap can be any appropriate cap that protects the oligo from degradation, e.g. a phosphorothioate bond between at least one of the last 2 nucleotides, or the last 3 or last 4 or last 5 nucleotides at the 5′ and/or 3′ ends.

Additive reactions can also be performed. In the embodiment depicted in FIG. 1B the fourth dsDNA molecule O14 has a variable sequence of 100 bp. Parallel reactions can produce a plurality of additional dsDNA molecules having complementary and/or overlapping sequences with the third dsDNA fragments that will form the fourth dsDNA molecule. The additional dsDNA can also have a variable sequence of, for example, 100 bp or any suitable length. Any of the dsDNA molecules can be cut with one or more restriction endonuclease(s) to produce a plurality of dsDNA fragments having 3′ and/or 5′ overhangs that contain complementary sequences at the 3′ and/or 5′ overhang of one or two other dsDNA fragments. Thus, in any embodiment and at any step the synthesis can include one or more dsDNA fragments that have a 3′ and a 5′ overhang sequence complementary to an overhang sequence on at least one other dsDNA fragment in the mixture. The overhangs can comprise at least a portion of the variable sequence of each dsDNA molecule. The dsDNA fragments can be combined to synthesize a much longer variable sequence in a product dsDNA molecule.

FIG. 2 provides a more detailed illustration of methods of the invention. Depicted are at least two oligonucleotides O1-O2 and an anchor strand O3, and paired oligonucleotides O4-O5 and paired anchor strand O6. FIG. 2B shows the complementary overlapping 3′ and 5′ overhang sequences that occur after restriction endonuclease digestion of the first and paired dsDNA molecules. The variable sequence is depicted as a 10mer and forming part of the 3′ or 5′ overhang sequences in the oligos. Also depicted is the second dsDNA molecule (O9) (illustrated with a variable sequence of 16 nucleotides) that is synthesized after annealing and amplification (PCR2) of the first and paired dsDNA fragments. In the embodiment depicted in FIG. 2, annealing, ligation 0 (L0), and PCR1 occur between the at least two oligonucleotides and anchor strands, here depicted in binding sets O1-O3 and O4-O6 to form the first (O7) and paired (O8) dsDNA molecules. Digestion with restriction endonuclease is then performed to produce first and paired dsDNA fragments followed by ligation 1 (L1) to additional dsDNA fragments (here the paired dsDNA fragment), then PCR2 to form the second dsDNA molecule, here depicted as having a variable sequence that is a 16mer (O9).

The second dsDNA molecule can then be digested with restriction endonuclease to form second dsDNA fragments, and ligated (L2) with an additional dsDNA fragment followed by PCR3 to form the third dsDNA molecule, which is depicted as having a 28mer variable sequence (O10, (FIG. 1B). The third dsDNA molecule can in turn be digested with restriction endonuclease and in one embodiment form third dsDNA fragments (e.g. O11), which can be combined and ligated (L3) with an additional dsDNA fragment (e.g. O12). One or more dsDNA fragments with a 3′ and 5′ overhang (e.g. 125, 130) can be included, and PCR3 performed to yield the fourth dsDNA molecule (O14), which is depicted as having a 100mer variable sequence. One or more additional dsDNA fragments (e.g. 125, 130, O12) can be included in the reaction, which can derive from multiplexed or parallel synthesis reactions. In this embodiment the additional dsDNA fragments are derived from an additional dsDNA molecule digested with a restriction endonuclease. Any dsDNA molecule can be synthesized with two restriction sites to produce a dsDNA fragment with an overhang at both the 5′ and 3′ end. A “parallel DNA synthesis reaction” can be a reaction for the synthesis of a DNA molecule of a distinct desired sequence, i.e. a different sequence than the primary reaction it is parallel to. The parallel reaction can be conducted as a separate reaction in a separate pool, but can also be a multiplexed reaction in the same pool. A DNA molecule of desired sequence from a parallel synthesis reaction can contain an overlap at the variable sequence with the DNA molecule of desired sequence in the primary reaction.

The terms “first dsDNA molecule,” “second dsDNA molecule,” “third dsDNA molecule,” “fourth dsDNA molecule,” “dsDNA fragments,” “additional dsDNA molecule,” and “paired dsDNA molecule” “first anchor strand,” etc., “paired anchor strand,” are relative terms that are provided to assist in tracking a molecule through any step(s) in the method, and do not necessarily refer to any absolute point or DNA molecule or fragment in the reaction. A “paired” dsDNA molecule or fragment contains a variable sequence that overlaps with and is at least partially complementary to the variable sequence of the reference dsDNA molecule or fragment (e.g. for at least 3 or at least 4 or at least 5 consecutive bps). In one embodiment a “paired” dsDNA molecule is multiplexed with a reference dsDNA molecule and an “additional dsDNA molecule” synthesized in a parallel synthesis. For example, the first dsDNA molecule contains a variable sequence and its paired dsDNA molecule can contain a variable sequence that at least partially overlaps with the variable sequence of the first dsDNA molecule, thus enabling them to be synthesized into a single, larger dsDNA molecule. In another embodiment the variable sequence of the first dsDNA molecule will at least partially overlap with the variable sequence of at least one additional dsDNA molecule. The second dsDNA molecule contains a variable sequence of the first and paired (or additional) dsDNA molecule, and in turn can at least partially overlap with a paired dsDNA fragment or additional dsDNA fragments having an at least partially complementary variable sequence. The third dsDNA molecule contains a portion of the variable sequence from the at least one second dsDNA molecule, and can further contain a portion of the variable sequence from the first dsDNA molecule, and can also have a variable sequence of one or more additional dsDNA molecules. The fourth dsDNA molecule can contain a variable sequence from the first dsDNA molecule (and its pair), second dsDNA molecule (and its pair), and third dsDNA molecule (and its pair); in some embodiments the fourth dsDNA molecule contains a variable sequence of a plurality of third dsDNA molecules. Such can continue and five to ten or more dsDNA molecules can be synthesized in hierarchal fashion, as generally depicted in FIG. 3. When digested by a Type IIS restriction endonuclease the dsDNA molecules will produce a dsDNA fragment having 3′ and/or 5′ overhang sequences that are complementary to at least one other dsDNA fragment in the mixture (or produced by a parallel synthesis reaction) at their variable sequences. In any embodiment any of the dsDNA molecules can be formed without the use of blunt end ligation. Thus, whether the first, second, third, fourth, paired, additional, etc. dsDNA molecules, any of the steps or methods can involve the use of annealing and not utilize blunt end ligation.

The methods therefore allow the production of a product DNA molecule having a variable sequence of any length without the need for a conventional oligonucleotide synthesizer, which typically relies on chemical synthesis (e.g. phosphoramidite chemistry). Instead, the methods can rely solely on enzymatic-based synthesis as depicted herein, and therefore the DNA molecules or polynucleotides can be produced on demand. DNA molecules can refer to single-stranded polynucleotides or double-stranded DNA bound by Watson-Crick base pairing. The methods can also involve performing multiple cycles of PCR or another DNA amplification procedure on any product DNA molecule. In some embodiments the methods can be performed on the polynucleotides using only enzymes, and buffers that support the enzymes.

In any embodiment the methods or any step of the methods can be performed without cloning or the need for cloning e.g. without the use of a host cell at any point in the method. In any embodiment the methods or any step of the methods can be performed entirely in vitro, or can be performed without the use of a living cell for any purpose in the method. In any embodiment the methods or any step of the methods can be performed without the use of terminal deoxynucleotidyl transferase (TdT), or without the use of a template independent DNA polymerase. In any embodiment the methods or any step of the methods can produce a scarless product DNA molecule. By scarless DNA is meant DNA that does not have any nucleotide(s) introduced by or from the process of synthesizing the DNA or nucleotides from which it is made, at least with respect to the variable sequence of the product DNA molecule (e.g. residue nucleotides from a linker, or adaptor, or flanking sequence). In any embodiment the methods or any step of the methods can produce a product DNA molecule that is barcode free, or free of a nucleotide sequence placed for identification of origin purposes. A barcode can be a sequence that is not otherwise needed but has a particular sequence and is used to identify a sequence of DNA. In various examples and embodiments a barcode sequence is 6-8 nucleotides in length, or 4-10 nucleotides in length. In any embodiment the methods or any step of the methods can be performed without any part of any oligonucleotide used in the method being immobilized, i.e. bound to a solid phase or solid support (e.g. a bead, DNA chip, microfluidic surface, etc). In any embodiment the oligonucleotides can be annealed in solution, and can be ligated in solution, i.e. without any oligonucleotide in the step or method being bound or partially bound to a solid phase or solid support (e.g. a DNA chip, bead, surface, or other solid phase.

In any embodiment the methods or any step of the methods can synthesize the product DNA molecule without the use of and without performing chemical assembly techniques (e.g. phosphoramidite chemistry). In any embodiment the methods can synthesize DNA molecules of desired sequence without the use of linker, adapter, or spacer oligonucleotides or sequences. “Linker,” “adaptor,” or “spacer” molecules can be short oligonucleotides that can be ligated to the ends of other DNA or oligonucleotide molecules. Linker, adaptor, or spacer molecules can also be used to provide for release of a polynucleotide from a solid support, or to link or tether a polynucleotide to a solid support. These molecules or sequences can provide sticky ends and/or overhangs allowing for ligation. Linker, adaptor, or spacer DNA sequences can also comprise, for example, recognition sites (e.g for an endonuclease), primer binding sites, polyU sequences, or can be a sequence having one or more uracil residues. Linkers, adaptors, or spacer DNA as referenced herein can be sequences that do not contain a nucleotide sequence that will be a part of the DNA molecule of desired sequence synthesized in the methods. In any embodiment of the methods the oligonucleotides or anchor strands that are used to synthesize the DNA molecule of desired sequence can have one or more of the above-described structures, but said structures are not provided on a separate linker, adaptor, or spacer molecule. In any embodiment the method can be conducted without the use of linkers, adaptors, or spacer DNA or sequences. In any embodiment of the methods the DNA molecule of desired sequence is synthesized using only oligonucleotides or anchor strands that contain at least a portion of the nucleotide sequence that will be present in the synthesized DNA molecule of desired sequence. In any embodiment the portion can be at least 6 or at least 8 or at least 10 or at least 16 or at least 28 or at least 50, or at least 100 consecutive nucleotides. This is a further advantage of the methods and makes the methods more suitable for automation.

In any embodiment the methods or any step of the methods can assemble the product DNA molecule using only enzymatic assembly of oligonucleotides. In any embodiment the methods or any step of the methods can be performed by drawing the at least two oligonucleotides and anchor strands from a library comprising less than 20,000 members, or from any library described herein. In any embodiment the at least two oligonucleotides and anchor strands can be selected from an oligonucleotide library having less than 10,000 members, or from any oligo library described herein. In any embodiment the methods or any step of the methods do not utilize or require the use of a vector in the methods.

The product DNA molecule can optionally be formed having conserved flanking sequences and/or, optionally having universal primer binding sites on the 3′ and 5′ ends of the product DNA molecule. The at least two oligonucleotides can be formed with one or more primer binding sites, which can provide binding sites for primers in amplification procedures (e.g. by PCR). Once the anchor strands are no longer necessary (e.g. a sufficiently long product DNA molecule has been synthesized), amplification can be done using primers that bind to the conserved flanking sequences and the universal primer binding sites are not needed.

The method can be facilitated by the use of recognition sites for a restriction endonuclease that can be effectively activated or inactivated. For example with reference to FIG. 1 O1 can have an inactive recognition site and O2 an active site programmed into the sequence when bound to the anchor strand O3. This recognition site can be utilized to digest the formed dsDNA molecules O7/O8 and digest the dsDNA molecules at one location leaving 5′ and 3′ overhangs. When multiplexing or in single synthesis reactions the restriction sites can be formulated within the sequences so that the restriction site is active on one side (e.g. the 3′ or 5′ side) of the variable sequence of a first dsDNA molecule and inactive on the opposite side, and vice versa for a second dsDNA molecule of the pair that will be combined in a synthesis step of the invention. Thus, when digested each dsDNA will produce two dsDNA fragments, which can then be annealed. However, in some embodiment where a larger dsDNA is arrived at (e.g. one having at least a 20mer or 28mer or similar variable sequence), and when the use of additional dsDNA fragments having a complementary 3′ and 5′ overhang 130 from parallel reactions is contemplated, the dsDNA molecule can be formulated so that it has active restriction recognition sites on both sides of the dsDNA molecule. Thus, when digested it will be cut into at least three fragments, at least one having both a 3′ and 5′ overhang sequence, which can be at the variable sequence. This additional dsDNA fragment can then be included within an annealing and ligation reaction with at least one 3′ end and at least one 5′ end of the dsDNA molecule, per FIG. 1B. This allows for the ability to greatly increase the length of the variable sequence in the product dsDNA molecule. The recognition (and restriction) sites can be turned “on” or “off” by utilizing a primer having a nucleotide mismatch, so the product dsDNA molecule no longer has an active recognition site (or does have one where formerly it did not). For example, the restriction site for BsaI is 5′-GGTCTC(N1)-3′ (SEQ ID NO: 17). By changing one nucleotide in the sequence one can activate or inactivate the restriction recognition site (e.g. turn the restriction site “on” or “off”). This can be accomplished by utilizing a primer with a single mismatch, thus changing the sequence produced. This can be utilized for any restriction endonuclease and can be used to place or remove a recognition site on either or both ends of the DNA.

In any embodiment the methods can include a step of removing conserved flanking sequences and/or primer binding sites on one side or both sides of the DNA molecule after amplification to yield a product DNA molecule. Methods of removing flanking sequences are known in the art. In some embodiments the conserved flanking sequences and/or primer binding sites can be utilized to add length to the product DNA molecule, or to surround the product DNA molecule with transcriptional elements (which can be on the 5′ and/or 3′ side of the variable sequence) or other beneficial sequences that will be utilized in the final desired sequence. For example, the flanking sequences can be set to provide a promoter in front of (e.g. 5′ to) the variable sequence, and/or to provide a terminator (i.e. regulatory sequences) after (or 3′) to the variable sequence. In one embodiment the product DNA molecule is a gRNA sequence (e.g. of 16-20 bp). The flanking sequences can optionally be set to provide a promoter in front of the gRNA sequence, and a Cas9 handle and terminator after it. Thus, in some embodiments the product DNA molecule can be expanded to encompass the primer binding sites and/or flanking sequences and/or one or more regulatory sequences and/or a Cas9 handle, any of which can provide more utility than being only binding sites for primers. In any embodiment of the methods the primer binding sites can be universal primer binding sites.

Any of the methods disclosed herein can be performed in an automated method, for example by an automated instrument. An automated method is one where no human intervention is necessary after the method is initiated—the method goes to completion from that point without a human having to perform any action. The automated instrument can contain components for selecting oligonucleotide members from the oligo library. A DNA sequence to be assembled can be uploaded, recorded on, or stored on a non-transitory computer-readable medium. A non-transitory computer-readable medium can be programmed to execute automated steps when inserted into or otherwise in electronic communication with a processor attached to or comprised within the automated instrument. The automated steps can be any disclosed herein for performing any method disclosed herein. Thus, the invention also provides a non-transitory computer-readable medium that is programmed with the locations of each member of an oligonucleotide library described herein, where the oligonucleotide library is present on a suitable support structure for the oligo library. In one embodiment the non-transitory computer-readable medium is programmed with the locations of at least 6,000 or at least 9,000 oligonucleotide library members. The medium can also be programmed with instructions to combine 4-6 members of a binding set from the library and to assemble the members of the binding set into a product DNA molecule according to the methods described herein. A “member” of a library is one or more polynucleotides at a location. An oligo library can be comprised on any type of medium, for example a multi-well plate or plurality of plates.

The invention also provides kits having an oligo library described herein located on a medium. The medium can be any suitable medium, for example one or more of a DNA chip, one or more bead(s), microtubes, one or more of a 96 well plate, one or more of a 384 well plate(s), one or more 1536-well plate(s), one or more microfluidic reaction support(s), one or more microtiter plate(s), one or more nanotiter plate(s), one or more picotiter plate(s), or other solid support or solid phase surface that can retain oligonucleotide members of the library. When more than one medium is utilized the media can be present in numbers sufficient to accommodate the oligo library. The medium containing the oligonucleotide library can contain members in any suitable volume, and examples include volumes of 1 nl up to 100 ul, or 10 nl up to 100 ul. A DNA chip (or DNA microarray) is a solid surface having a collection of microscopic locations, to which oligonucleotides can be attached and/or stored.

The methods of the invention can synthesize a product DNA molecule having a very low error rate. In various embodiments the methods can produce any product DNA molecule described herein with error rates of less than 1 in 1,000 base pairs, or less than 1 in 2,000 base pairs, or less than 1 in 2,400 base pairs, or less than 1 error in 2,500 base pairs, or less than 1 error per 3,000 base pairs, or less than 1 error per 5,000 base pairs, or less than one error per 5,300 base pairs, or less than 1 error per 6,000 base pairs, or less than 1 error per 8,000 base pairs, or less than 1 error per 12,000 base pairs, or less than 1 error per 14,000 base pairs.

### General Steps

In any embodiment the methods can begin with a pooling of at least two oligonucleotides and an anchor strand, e.g. from the oligo library. A general embodiment is depicted in FIG. 1A-1B. In the embodiment described here multiplexing will be utilized, and the at least two oligos and anchor strand have been prepared with restriction sites for BsaI, although any restriction enzyme or Type IIS restriction enzyme can be utilized.

The pool of oligos can be subjected to a step of annealing and a step of ligation (e.g. L0 and PCR1). The ligation step can be performed by contacting the pool of oligonucleotides with a ligase, for example T4 DNA ligase. But any ligase can be utilized at any step in the invention. Ligation can be preceded by the annealing of complementary 5′ and 3′ overhang sequences on the dsDNA fragments produced by the digestion with restriction endonuclease. Ligation can also involve contacting the oligos with a ligase, and the formation of a covalent bond between adjacent nucleotides. The polymerase chain reaction (PCR) is a common reaction in biology known to persons of ordinary skill. PCR can be used in the invention according to normal procedures and well known techniques. PCR (PCR1) results in amplification of the oligos, depicted in the example in FIG. 1A as O7 and O8 and having variable sequences that are 10mers. The methods can involve a step of digestion with restriction endonuclease and annealing with additional dsDNA fragments and ligation, followed by a step of PCR (D1 and PCR2). The oligo set can be digested with the restriction enzyme (e.g. a Type II restriction endonuclease). Digestion results in dsDNA fragments with 3′ and 5′ overhangs, which can then annealed to other fragments having complementary overhangs, ligated, and amplified in the PCR2 step to form dsDNA molecules having variable sequence(s), depicted in the embodiment in FIG. 1A as a 16mer (O9). Another digestion (DL2) can be performed on the product to result in dsDNA fragments, steps of annealing, ligation, and PCR3 to form dsDNA molecules, depicted in FIG. 1A as having variable sequences that are 28mers ((O10, FIG. 1B). A step of digestion with restriction endonuclease, annealing with additional dsDNA fragments and ligation (DL3) can then be utilized. The ligation can optionally involve dsDNA fragments from parallel reactions or otherwise synthesized that have a 3′ and/or 5′ overhang complementary to at least one other dsDNA fragment in the mixture. Thus, optionally the annealing step can involve the addition or use of additional dsDNA fragments that have 3′ and 5′ overhang sequences having a complementary sequence to two other dsDNA fragments in the mixture. Annealing of the mixture of dsDNA fragments results in a longer dsDNA molecule. In this manner the length of the variable sequence can be quickly increased. In this example the product dsDNA molecule (O14) is from the combination of two dsDNA fragments and two additional dsDNA fragments, all from 28mers. The product dsDNA molecule of desired sequence in this embodiment is a 100mer variable sequence depicted in FIG. 1B.

### Primer Binding Sites

The primer binding sites can be present on some DNA molecules in any embodiment of the methods. In some embodiments the sites can be present on the at least two oligonucleotides and on a product dsDNA molecule (e.g. the first dsDNA molecule). Primer binding sites can be part of the conserved flanking sequences, or distinct from them. However, in some embodiments the distinct primer binding sites can be eliminated in any step after the anchor strand is no longer utilized. For example, the sites can be eliminated after formation of the at least one first or second dsDNA molecule, and a portion of the conserved flanking sequences used as primer binding sites thereafter. Thus, the at least two oligonucleotides can have primer binding sites, which then are present in the at least one first dsDNA molecule, but any one or more of the second, third, and fourth dsDNA molecules can lack (or can have) primer binding sites. Primer binding sites can also be added to dsDNA molecules at any step where convenient in the methods, e.g. on forming the final product dsDNA molecule it may be found desirable to have a convenient methods of amplifying the product. The length of the primer binding site and/or of the complementarity between the primer and primer binding site can be at least 4, or 5, or 6 nucleotides or at least 10 or at least 15 or at least 18 or at least 20 nucleotides or at least 25 nucleotides, or less than 15 nucleotides, or less than 12 nucleotides or less than 10 nucleotides or less than 8 nucleotides, which in any embodiment can be consecutive nucleotides. But no particular length is necessary, only that the primer binding site allow for binding of a primer and amplification of the molecule. In any embodiment the primer binding sites can be universal primer binding sites and can have the same sequence on all molecules in a mixture having primer binding sites, thus enabling amplification of the mixture from a single set of primers. In any step of amplification all dsDNA molecules to be amplified can have a universal primer binding site of the same sequence. In any embodiment the at least two oligonucleotides or DNA molecule of desired sequence can have a single (i.e. only one) primer binding site on the 3′ and/or 5′ ends.

In any step or embodiment of any of the methods one, or a plurality, or all of the primer binding sites on the polynucleotides used in the methods can be universal primer binding sites. Universal primers are complementary to and can bind to a universal primer binding site. Universal primers serve to permit one or a small set of primers to perform amplification and assembly on an entire mixture or pool of oligonucleotides, or on a sub-set of oligonucleotides. “Universal primer binding sites” can be a primer sequence in common to a particular set of oligonucleotides or DNA molecules. For example, in various embodiments a universal primer binding site may exist on at least 25%, or at least 50%, or at least 60%, or about two-thirds, or at least 70% or at least 80% or at least 90% or at least 95% or at least 98% or 100% of the polynucleotides in a particular mixture or pool. In some embodiments primer binding sites (including universal primer binding sites) can be located only on the terminal 50 nt of a DNA molecule on either or both ends, or only on the terminal 30 nt, or 25 nt, or 20 nt or 15 nt. But in other embodiments primer binding sites (including universal primer binding sites) can be located on a conserved flanking sequence. In any embodiment the primer binding site (including universal) can be not located on the variable sequence. In other embodiments of the methods it may be desirable to utilize ordinary primers or non-universal primers. While this will necessitate the use of additional primers and complementary primer binding sites to perform amplification and assembly, it can also give flexibility to the methods when desired. In some embodiments primer binding sites can be found on only one sequence of oligonucleotide (or its complement). Ordinary primers and primer binding sites differ from universals only in that they are not found on a large portion of sequences in the methods. The term “pool” of oligonucleotides is used herein according to the ordinary meaning indicating oligonucleotides in a distinct and separate reaction pool.

### Variable Sequence

As the methods proceed, whether performed in multiplex fashion or in parallel, the variable sequence in the dsDNA molecule can grow longer as the methods proceed due to progressively or serially combining more DNA and/or oligonucleotides containing a variable sequence that will be part of the product dsDNA molecule. In any embodiment the length of the variable sequence in the first dsDNA molecule can equal the length of the variable sequences from the at least two oligonucleotides combined and annealed on the anchor strand. In any embodiment the variable sequence in the first dsDNA molecule can be 6-14 base pairs, or 7-13 base pairs, or 8-12 base pairs, or about 10 base pairs, which can be adjusted depending on the dsDNA molecule to be synthesized. In any embodiment the second dsDNA molecule can have a variable sequence of 8-24 or 10-22 or 12-20, or 14-18 or 15-17 base pairs (or, as in any step, the length of the variable sequences in the dsDNA fragments from which it is synthesized, minus overlapping nucleotides). In any embodiment the third dsDNA molecule comprises a variable sequence of 18-38 or 20-36 or 24-32 or 26-30 or 27-29 base pairs. In any embodiment the fourth dsDNA molecule can have a variable sequence of 70-130 or 80-120 or 90-110, or 70-200 base pairs. But the length of the variable sequence in any step is not fixed and can be varied to whatever is convenient or desirable in the application.

The variable sequences can be sequences that will be present in the product dsDNA molecule, or that form the “desired sequence” of the DNA molecule of desired sequence, and that do not form part of a primer binding site or conserved flanking sequence. Thus, the variable sequence is an essential part of the DNA molecule of desired sequence. Thus, the variable sequences will vary in each construct depending on what portion of the final product DNA molecule it is carrying and what product dsDNA molecule is being synthesized. The product DNA molecule can be the DNA molecule having the desired sequence. In one embodiment all of the variable sequences in the at least two oligonucleotides will be present in the product dsDNA molecule produced at the end of whichever method is performed.

The variable sequence in the at least two oligonucleotides (or in any step or molecule of the methods) can be at least 4 nucleotides, or at least 5 nucleotides, or at least 6 nucleotides, or at least 10 or at least 12 or at least 15 or at least 18 or at least 20 nucleotides, or 3-7 nucleotides or 4-6 nucleotides, or 4-8 nucleotides, or 6-10 nucleotides, or 6-12 nucleotides, or 12-16 nucleotides, or 14-18 nucleotides. The variable sequence for an anchor strand can be equal to the lengths of the variable sequences in the at least two oligonucleotides. In any embodiment the variable sequence on the anchor strand can anneal entirely with the variable sequences on the at least two oligonucleotides.

In any embodiment the variable sequence can be present as one consecutive sequence. In other embodiments the nucleotides of the variable sequence can be separated singly or in groups of two or three or four or more consecutive nucleotides throughout the oligo sequence to comprise a variable region. The variable sequence can be at least a portion of the desired sequence or product dsDNA molecule to be synthesized in the methods. In one embodiment the library can contain a distinct oligonucleotide for each possible variable sequence of an oligonucleotide, and each distinct sequence can be present at a distinct location in the oligo library. Thus, each oligo having a distinct variable sequence can be located at a distinct location in the oligo library. For example, O1 of the at least two oligonucleotides has a variable sequence. When the variable sequence is five nucleotides, O1 can have 1024 possible nucleotide sequences, i.e. 4×4×4×4×4 equals 1024 variable sequences for O1, each of which can be present at 1024 distinct locations in the library. The same is true for O2-O6 as depicted in the embodiment of FIG. 1. In various embodiments the variable sequence can be the sequence that defines each distinct location in the library for each of the “at least two oligonucleotides.” In various embodiments one-half (or only a portion) of the variable sequence is passed to the next step in the methods, with the remainder of the variable sequence being provided by the dsDNA fragment with which the present fragment is combined or annealed.

In any embodiment the variable sequences of two dsDNA molecules (including produce dsDNA molecules) can overlap, i.e. have a complementary sequence for two or more nucleotides. In some embodiments any two dsDNA molecules can contain variable sequences that overlap by at least 1 or at least 2 or at least 3, or at least 4, or at least 5, or at least 6, or at least 7, or at least 8 nucleotides, or by 1-6 nucleotides or 2-4 or 2-5 nucleotides, or by 3-10 nucleotides, or by about 4 nucleotides, or by at least 10 nucleotides, or by more than 8 nucleotides. In various embodiments the first or second or third dsDNA molecules, or additional dsDNA molecules described herein can have variable sequences that overlap with the variable sequences of other dsDNA molecules as described. For example, the first dsDNA molecule can have a variable sequence that overlaps with the variable sequence of its paired dsDNA molecule or an additional dsDNA molecule, the second dsDNA molecule, third dsDNA molecule, or additional dsDNA molecules can all similarly have variable sequences that overlap with the variable sequences of their paired or additional dsDNA molecules. But dsDNA molecules can also have variable sequences that overlap with the variable sequence of any other dsDNA molecule (e.g. a second dsDNA can be made to overlap with a third dsDNA from a parallel synthesis reaction.

In any embodiment dsDNA fragments can also have a 3′ and/or 5′ overhang sequence that contains a variable sequence that overlaps by at least 3, or at least 4, or at least 5, or at least 6, or at least 7, or at least 8 nucleotides, or by at least 10 nucleotides, or by more than 8 nucleotides with the variable sequence overhang of other (paired) dsDNA fragments. Thus, a first dsDNA fragment can have a variable sequence on the 3′ and/or 5′ overhang that overlaps with that of a paired dsDNA fragment on its 5′ or 3′ overhang. A second dsDNA fragment can have 3′ and/or 5′ overhang sequence that contains a variable sequence that overlaps with that of its paired dsDNA fragment, or an additional dsDNA fragment. The 3′ and/or 5′ overhangs can be produced by restriction endonuclease action on a dsDNA molecule, and can also be synthesized separately and provided to any of the reactions. In any step of the methods dsDNA fragments can have 3′ and/or 5′ overhang sequences that are part of the variable sequence, and which can be used to anneal and combine them with one or more other dsDNA fragments at their variable sequences.

### Degenerate Nucleotides

One or more of the at least two oligonucleotides and/or the anchor strands used in the methods can optionally have one or more degenerate nucleotides. In one embodiment only anchor strands contain degenerate nucleotides. Degenerate nucleotides refers to nucleotides present at degenerate positions in the oligo sequence. In any embodiment the degenerate nucleotides can be present within and as part of the variable sequence of the anchor strand or other oligonucleotide in the methods. A degenerate nucleotide in an oligo is a nucleotide that can be any of A, C, T, or G, i.e. a nucleotide position in a library member that has been randomized. Randomization can be performed by simply supplying sequences with all four bases during oligo synthesis, thus producing an oligonucleotide with randomized positions. However, in some embodiments a degenerate nucleotide can be a universal base, which can base pair with all four of the standard bases. Examples include deoxy-inosine, 2-deoxyinosine, nitroindole, 5-nitroindole, 2′-deoxynebularine, 3-nitropyrrole, dP, dK, or other universal bases can be used to reduce degeneracy. 3-nitropyrrole 2′-deoxynucleoside, and 5-nitroindole 2′-deoxynucleoside can also be used as degenerate bases. An oligo having one or more degenerate nucleotides is a degenerate oligonucleotide. Degenerate oligos can be co-located at the same (degenerate oligonucleotide) location in an oligo library. Degenerate oligos can thus be present at a location as a group of slightly different sequences, with each degenerate oligo having a distinct sequence due to the degenerate nucleotides, yet all co-located at the same location. In some embodiments degenerate nucleotides on one oligo can anneal to nucleotides of a variable sequence on another (target) oligo, such as is depicted in FIGS. 1-2. In various embodiments any of the oligonucleotides in a binding set can have degenerate nucleotides in its variable sequence. In some embodiments at least one oligonucleotide in a binding set has degenerate nucleotides. In some embodiments only the anchor strand has a variable sequence containing degenerate nucleotides. With reference to FIGS. 1-2 the anchor strands are depicted as having degenerate oligonucleotides (designated “N”) in the variable sequence. A “binding set” is a group of oligonucleotides that bind to one another at a step in the methods disclosed herein, and form a dsDNA product in the methods. Thus, O1-O3 is a binding set as depicted in FIG. 1A, as is O4-O6. In some embodiments oligos of a binding set can bind substantially to one another (i.e. not merely by a small amount). In another embodiment oligos of a binding set bind to each other without mismatched base pairs. In some embodiments a binding set includes at least one oligo that binds completely to one, or two or more other oligos in the binding set. In various embodiment at least one oligo of a binding set binds at least 80% or at least 90% or at least 95% or 100% to one, or two or more other oligos of the binding set, i.e. no with no unmatched bases. A “target” oligo is a second oligo that a first oligo is intended to bind to in the methods.

In any embodiment the methods can involve a step of annealing of two (and optionally only two) oligonucleotides to a third oligonucleotide (e.g. one anchor strand). The two (and only two) oligonucleotides can bind to the same third oligonucleotide. The methods can involve a step of PCR on the annealed oligonucleotides to form a dsDNA molecule. The two oligonucleotides can have a variable sequence at a 3′ and/or 5′ end, a primer binding site at the opposite 5′ and/or 3′ end, and a conserved flanking sequence in between the variable sequence and the primer binding site.

One or more anchor strands in a method can have 3 or 4 or 5 or 6 or 7 or 8 or 3-5 or 3-6 or 3-7 or 3-8 or 4-5 or 4-6 or 4-7 or 4-8 or 6-10 or more than 8 or more than 10 or more than 12 degenerate nucleotides in its variable sequence. The one or more degenerate nucleotides in an oligo can be present as one consecutive sequence to comprise a degenerate sequence, or the degenerate nucleotides can be separated singly or in groups of two or more consecutive degenerate nucleotides throughout the oligo (e.g. an anchor strand). In some embodiments degenerate nucleotides are present only within a variable sequence of the oligos, or only within the variable sequence of the anchor strand(s). In one embodiment 60% or less or 70% or less of the nucleotides in the variable sequence of an anchor strand are degenerate oligonucleotides.

Degenerate oligonucleotides present at a location in the oligo library have multiple sequences at the location and can be grouped together and considered as one member of the library. For example, an anchor oligo (or other oligo) having, for example, five degenerate nucleotides can have 1024 possible sequences (4×4×4×4×4=1024), but all 1024 sequences can be co-located at a single defined location in the library. A location in the oligo library containing the multiple sequences of degenerate oligonucleotides is termed a degenerate oligo location. Multiple degenerate oligonucleotides (each of slightly different sequence) can be co-located at a single location in the oligo library. While in some embodiments all possible sequences of a degenerate oligonucleotide are provided at the same location (e.g. all 1024 possible sequences of a degenerate oligo having 5 degenerate nucleotides), in other embodiments multiple degenerate oligonucleotides can be located in groups of convenient numbers at multiple different locations in the oligo library. Degenerate nucleotides allow the user to therefore greatly reduce the number of positions in the oligo library. However, in some embodiments the degenerate oligonucleotides at a location can contain universal bases, and thus can have a “single” or smaller number of sequences at that location, even though the location is a degenerate oligonucleotide location. In some embodiments the number of degenerate oligonucleotides at the location can be reduced due to a number of the degenerate nucleotides being universal nucleotides.

Thus, while oligos having one or more degenerate nucleotides can be co-located together at a single defined location in the library, oligos having variable sequences with no degenerate nucleotides can each have their own defined location in the library, i.e. a separate location for each sequence. An oligonucleotide having one or more degenerate nucleotides can be co-located at a single location with all possible sequences of the oligo for each degenerate position present at the single location. In one embodiment only anchor strands have degenerate nucleotides and the “at least two oligonucleotides” do not.

For illustration, consider anchor strand O3 in FIG. 1 having ten variable nucleotides, including six degenerate nucleotide locations. Ten nucleotides of variable sequence would normally require over 1 million locations, but 03 has six degenerate nucleotides. Thus, O3 with 4 non-degenerate nucleotides can be present at locations L1 . . . . L256 for O3 (4×4×4×4) in the library, with each location containing the specific sequence for the non-degenerate portion of the variable sequence. And the 256 locations can have a group of oligos that provide all possible sequences for the degenerate nucleotides D1 . . . D4096 (4×4×4×4×4×4 or 4096). Thus, degenerate sequences D1-D4096 can all be present at each of variable locations L1-L256 for O3, with each location having a distinct sequence for the non-degenerate positions on the sequence, and oligos of all possible sequences at the degenerate positions. Thus, at location L1 for the example O3 can have variable sequence SEQ ID NO: 3 NNNACTCNNN (V1), having the non-degenerate portion of variable sequence and an oligo for all possible sequences for the degenerate nucleotides in each location (D1-D4096). At location L2 for O3, degenerate sequences D1-D4096 will all have non-degenerate sequence V2. At location L3 for O3 degenerate sequences D1-D4096 will all have non-degenerate sequence V3, and so on. Thus, degenerate sequences D1-D4096 for O3 are all present at locations L1-L256 for O3, with each degenerate sequence having the non-degenerate portion of the variable sequence. All anchor strands will thus contain the same set portion of the sequence, but all will vary in sequence at the degenerate nucleotides. Thus, the library can have 256 locations for O3 in this example.

### Oligonucleotide Library

The invention also provides methods of synthesizing a product DNA molecule from a library of oligonucleotide members according to the methods disclosed herein. The library of oligonucleotide members can have fewer than 10,000 or fewer than 5,000 oligonucleotide members (or locations), and the oligonucleotide members in the library are sufficient to assemble any possible polynucleotide sequence. The method involves assembling oligonucleotide members from the library to obtain the product DNA molecule.

With reference to FIG. 1A all oligonucleotides, O1-O6, are members in the library. O1-O6 can each have one variable sequences. The oligonucleotide library can be comprised on any one or more of a DNA chip, solid support, solid phase, bead, microfluidic surface, cell culture plate (e.g. 96 well, 384 well, or 1536 well), etc, or other structure where oligonucleotides can be stored at defined locations and be available for retrieval and use in the methods. In some embodiments the library will contain a distinct location for each of the possible variable sequences of O1-O6. In other embodiments degenerate nucleotides are used on one or more of the polynucleotides.

When O1-O2 and O4-O5 have a variable region having 5 variable nucleotides, the number of locations to accommodate the possible sequences of the oligos is 4 to the 5th power, thus 4×4×4×4×4 equals 1,024. Thus, in some embodiments there is a defined oligo sequence at 4,096 defined locations, with a single or unique defined variable sequence for O1-O2 and O4-O5 present at each of the locations. Thus, O1 oligos can have five variable nucleotides and thus 1024 possible sequences, which can be present at 1,024 defined locations for O1 with a single defined variable sequence at each location, and similar for O2 and O4-5.

Adding anchor strands O3 and O6 in this example, each anchor strand has four non-degenerate nucleotides, and six degenerate nucleotides. Thus, the library can also have 256 locations for each of O3 and O6 to accommodate oligos having non-degenerate nucleotides, with each location having a distinct sequence for non-degenerate nucleotide sequences. Additionally, each of the 256 locations can also have all possible degenerate sequences, thus 4,096 degenerate oligo sequences are present together at each of the 256 locations for the set nucleotides of the variable sequence. This example thus gives a total of only 4,608 distinct locations in the entire library (4×1024+2×256=4,608), from which one can assemble all possible DNA sequences. Even doubling the library size to accommodate a parallel synthesis gives only 9,216 members.

A location in the oligo library can be a well of a plate, a tube, or any other structure or force that segregates an oligonucleotide member in a distinct location, spatially separated from other members of the library sufficiently for it to be accessed individually and as a species at this distinct location.

The oligos can be maintained in their distinct locations as a single molecule (which can be amplified) or as a multiple copies of the same molecule from which a small volume can be taken and used in synthesis procedures. The distinct locations of each sequence can be identifiable to a software program that can be configured with a mechanical gantry or device that retrieves specific library members from the distinct location for use in a method of the invention where the defined oligonucleotide library member is required. In one embodiment an oligo library can be located in a collection of assay plates or small tubes, each containing a member of the oligo library, and to which instrumentation components can go and retrieve an oligo library member according to software instructions, which can be located on a non-transitory computer-readable medium. The non-transitory computer readable medium can also contain programmed instructions and/or steps for synthesizing a product DNA molecule according to any of the methods disclosed herein, and the programmed instructions and/or steps can be provided to an instrument in communication with the computer-readable medium. The programmed instructions or steps can direct the instrumentation to perform the assembly of a DNA molecule of pre-defined sequence according to any method disclosed herein, or to perform any of the methods provided herein.

Members of the oligonucleotide library are present at distinct locations, spatially separated from other members of the library. Thus, a member of the library can be a specific sequence present at its location (either a single or multiple copies). A non-degenerate oligo can have one sequence present at its library location. When degenerate sequences are used, the member of the library containing degenerate sequences can contain all possible degenerate sequences of that oligo member (or in some embodiments a subset of all possible sequence) in view of the number of degenerate nucleotides on the oligo, and present at a distinct location. Thus, in a library location of a non-degenerate nucleotide sequence the member of the library can contain one sequence. At a library location of a degenerate nucleotide sequence the member can contain multiple sequences, including a sequence for all possible sequences of the oligo in view of the degenerate nucleotides in the oligo sequence.

In some embodiments there can be a number of sequences of the all possible sequences that are not of interest. Thus, only a subset of all possible degenerate sequences need be present at the distinct location in a defined category library to assemble all possible sequences of interest. In any embodiment the distinct location can be defined by any suitable technique, for example reference points in a microscopic picture or grid of the solid support containing the oligo library. In some embodiment the distinct location of any or all oligo sequences can be stored on and/or communicated by a non-transitory computer-readable medium.

DNA with Overhangs

The product DNA molecules of any synthesis method disclosed herein can be assembled, if desired, into larger product dsDNA molecules. In some embodiments the product dsDNA molecules of any of the methods can be double-stranded blunt end DNA. DNA molecules can be synthesized so that the variable sequences between product dsDNA molecules contain an overlapping sequence. A product dsDNA molecule can be digested with a restriction endonuclease that cleaves within the variable sequence and leaves 3′ and/or 5′ overhang sequences or “sticky ends” in the resulting dsDNA fragments. These overhang sequences can overlap with (and be complementary to) nucleotides in the overhang sequences of another digested dsDNA fragment. These overhang sequences can then be used to assemble the dsDNA fragments into a larger DNA molecule through annealing of the complementary 3′ and/or 5′ sequences. In other embodiments the product dsDNA molecules can be synthesized having single-stranded overhang sequences of one or more nucleotides, or of 4 nucleotides or 5 nucleotides or 6 nucleotides or 7 nucleotides or 8 nucleotides or more, or 9 or 10 nucleotides, or more than 10 nucleotides, and provided as additional dsDNA fragments. Oligonucleotides or dsDNA fragments can then be annealed and joined using the overlapping or complementary nucleotides within these overhangs.

### Restriction Recognition Sites

Type IIS restriction enzymes cleave DNA at a defined distance from their recognition site and leave a 5′ and/or 3′ single-stranded overhang. The recognition site can be provided to lie outside of the variable sequence, and the cleavage site can be provided to lie within the variable sequence, leaving 3′ and/or 5′ overhangs on the resulting dsDNA fragments. Type IIS restriction endonucleases also find application in the invention for producing additional dsDNA fragments having single-stranded overhangs. The single-stranded overhangs can be present at the 3′ and/or 5′ ends, depending on where in the molecule the dsDNA fragment is to be positioned relative to other fragments. dsDNA molecules can be programmed or synthesized to have active recognition sites on the 3′ and/or 5′ sides of the dsDNA molecule and on one or both sides of the variable sequence. The dsDNA molecules can also be programmed to have cleavage sites within the variable sequence, or towards the 5′ and/or 3′ ends. dsDNA fragments can be joined by annealing dsDNA fragments having complementary overhanging 3′ and/or 5′ sequences and ligating to form a longer DNA molecule. Multiple additional dsDNA fragments having 3′ and/or 5′ overhangs (e.g. 125 and 130 in FIG. 1B) can be annealed to dsDNA fragments having complementary 5′ and/or 3′ overhangs (e.g. O11 and O12). Thus, dsDNA fragments at any step can be annealed to one or more additional dsDNA fragments to more rapidly advance the size of the variable sequence of the product dsDNA molecule. In this hierarchal fashion a dsDNA molecule can be synthesized having an about 100 bp variable sequence or larger (e.g. as illustrated in FIG. 1B and FIG. 3).

In any embodiment the restriction enzyme utilized in the invention can be a Type IIS restriction enzyme. In one embodiment the Type IIS restriction enzyme is one that only cleaves dsDNA. In one embodiment Type IIS restriction sites can be encoded into the conserved flanking sequences, as illustrated in FIG. 1. Any Type IIS restriction enzyme can be utilized. In various embodiments the restriction sites can be BsmBI sites, or BsmBi sites, or EciI sites, or BspMI sites, or FauI sites, etc. BsmBI recognizes the sequence 5′-CGTCTC(N)-3′ (SEQ ID NO: 16). The enzyme generally cleaves to the 3′ side of N. BsaI is another Type IIS restriction enzyme, which recognizes the sequence 5′-GGTCTC(N1)-3′ (SEQ ID NO: 17) and generally cleaves to the 3′ side of N. Persons of ordinary skill with resort to this disclosure will realize many other Type IIS restriction enzymes can be utilized in the invention. Such persons can also encode recognition sites in the CFSs for particular restriction endonucleases so that they will cleave within the variable sequence and provide overhanging nucleotides. In any embodiment of the methods disclosed herein any of the DNA molecules utilized in or produced by the methods can contain one or more Type IIS restriction endonuclease recognition sites. In various embodiments the restriction enzymes can leave an overhang of at least 2 bp, or at least 3 bp, or at least 4 bp.

In some embodiments of the methods restriction recognition sites on the dsDNA molecules can be turned “on” or “off” as needed. While dsDNA molecules can comprise a restriction recognition site (e.g. on the conserved flanking sequence (CFS)), the CFS sequence can be changed in the method, thereby replacing the restriction recognition site with a sequence not recognized by the enzyme. This can be accomplished by utilizing in an amplification step a primer having at least one base mismatch with the site on the CFS. During amplification use of the mismatched primer results in a change in the sequence during amplification and thereby deactivates the recognition site. Thus, at a point in the method when the site is no longer useful the sequence of the recognition site can be modified during amplification using a primer mismatched at the recognition site, thus modifying the sequence in the amplification product and effectively turning the recognition site “off”. The primer can be mismatched to the sequence on the recognition site, or a sequence near the recognition site that encompasses at least a portion of the recognition site. The primer can mismatch the recognition site at at least one nucleotide, or at one nucleotide, or at two nucleotides, or at three nucleotides.

### DNA Data Storage

DNA is stable even over periods of thousands of years and even in many extreme environments, giving it great advantages for use in storing information. Any of the methods disclosed herein can be applied to encoding digital data into DNA. One or more product DNA molecule(s) can have a sequence that comprises an encoded non-genetic message. One or more product DNA molecule(s) can have a sequence that corresponds to bytes of information that encode the non-genetic message. The bytes of information can be decoded with reference to a coding scheme or key that assigns one or more letters, words, characters, or numbers to each encoded byte of information. A non-genetic message can be, for example, a word, a phrase, an identifying watermark, textual information, the contents of a book or library of books, or any other information that can be provided in a reference language.

For example, as illustrated in FIG. 5, a DNA molecule having a 16 bp variable sequence can be synthesized and easily accommodates four bytes of information on the variable sequence, where each byte is encoded by an assigned sequence of nucleotides. In this example a four nucleotide sequence represents a byte of information, which can correspond to a character or symbol (e.g. a letter, numeral, or other symbol). Thus, in this example 256 characters can be encoded in each byte of information (4×4×4×4). Thus, the alphabet of any language in the world can be easily accommodated within these 256 bytes of information and a sufficient number of numerals and other numbers or other characters utilized in communication as well. In various embodiments the non-genetic message can be encoded in a reference language, for example, English, French, German, Italian, Spanish, Latin, Japanese, Hindi, Chinese, Russian, or any language. A reference language can also include numbers and special characters, even though not formally part of the reference language. But any information can be encoded in the DNA sequence in any language. In various embodiments the message can be at least 100 characters long, or at least 500 characters long, or at least 1000 characters long, or at least 10,000 characters long. Nucleotides having non-standard bases can also be used, which can expand the number of characters available.

The product DNA can also encode a character (e.g. a letter, a word, a number, a punctuation mark, word character, or other characters utilized in communication) that indicates where in the sequence the information encoded by that DNA molecule is to be placed. FIG. 5 depicts 16 bp product DNA molecules having four bytes of four nucleotides each. The last byte in each product DNA sequence indicates the location in the message where the preceding three bytes are placed; this is conveniently a numeral but can be any character that can be placed into a definable sequence. While a 4 nucleotide byte provides up to 256 identifiers the byte can be any convenient length of nucleotides. For example, bytes can be comprised of 3 nucleotides, or 5 nucleotides or 6 nucleotides (allowing for 4,096 identifiers), or 7 or even 8 nucleotides, or more than 8 nucleotides, allowing for many more identifiers to be included. Limited numbers of identifiers can also be expanded by placing DNA molecules in a single well up to the number of identifiers, and then assembling the messages from the DNA in the order of the sequence of wells. Using this method with only 4 nucleotide bytes even a single 384 well plate can contain over 98,000 DNA molecules (256 molecules×384 wells), which can be assembled in order to provide almost 300,000 bytes of information (in addition to the identifier), providing for over 153 million words, or over 550,000 pages of text utilizing a standard page and 512 words/kb, in a single 384 well plate. When a five nucleotide byte is used over 1,024 molecules can be individually identified times 384 wells, i.e. 393,000 molecules, or over 1 million bytes of information in a single plate. Multiple plates can be used to accommodate much greater amounts of information. Therefore, an unlimited amount of information can be encoded and stored indefinitely according to the methods. Because of the high stability and small size of DNA, entire libraries of information can be encoded according to the methods.

Thus, the invention provides methods of storing data in a DNA sequence, which can involve determining a sequence of DNA that encodes a non-genetic message according to a coding scheme that can translate the non-genetic message from a reference language into a DNA sequence and vice versa; synthesizing the sequence of DNA that encodes the non-genetic message according to a method disclosed herein; and thereby store data in a DNA sequence. The method can optionally be repeated until the non-genetic message is recorded in the sequence.

A coding scheme is a set of codes (e.g. 4 or 3 nucleotide codons, an example of which is shown in FIG. 5) that assign a particular character of a reference language to a particular codon. For example, the standard DNA codon table is a coding scheme, but it may be advantageous to use a coding scheme that is not easily transcribable. Examples of coding schemes are known to persons of ordinary skill in the art, e.g. any of those disclosed in U.S. Pat. No. 10,818,378, which is hereby incorporated by reference in its entirely, including all tables, figures, and claims; or Marillonnet et al., Nature Biotech., Vol. 21, pp. 224-226 (2003). But many such coding schemes are known and new coding schemes can be easily devised by skilled persons. After synthesis of dsDNA molecules according to any method described herein the dsDNA molecules can be combined using additional DNA joining techniques known in the art to build a much larger dsDNA molecule, which contains the encoded information and can be stored indefinitely. Thus, in one embodiment a non-genetic message can be provided and translated according to a coding scheme into a DNA sequence, which can be synthesized, thereby storing the data in a DNA sequence.

### CRISPR Guide RNA

The invention can also be applied to the synthesis of guide RNAs (gRNA) for use in CRISPR-Cas9 methods. Using the methods any sequence of gRNA can be quickly constructed. Guide RNA constructs can also be constructed from oligonucleotides in the oligonucleotide library. A product DNA molecule can be synthesized in the methods having a DNA sequence that encodes an initial guide structure. The initial guide RNA structure can encode a gRNA with the necessary prokaryotic or eukaryotic transcriptional elements for in vitro transcription in proper order, for example any one or more of a promoter, a sequence of gRNA, and a terminator. In some embodiments the gRNA can encode a Cas9-binding hairpin (Cas9 handle). In some embodiment the transcriptional elements include a promoter and/or a terminator. In some embodiments the product DNA molecule can encode 20 bases for the gRNA. FIG. 6 depicts one embodiment in which a dsDNA molecule is synthesized into an initial guide structure having the transcriptional elements. In any of the methods disclosed herein the product dsDNA molecule can encode a guide structure or gRNA or other RNA molecule. Since all possible polynucleotide sequences can be assembled from the oligo library, any initial guide structure or gRNA or RNA can be assembled in the methods.

### EMBODIMENTS

In one embodiment the method involves annealing at least two oligonucleotides of about 30-60 nucleotides in length with an anchor strand about 30-70 nucleotides in length according to the methods disclosed herein.

In another embodiment the method involves annealing at least two oligonucleotides of about 40-50 nucleotides in length with an anchor strand about 40-50 nucleotides in length according to the methods disclosed herein.

In another embodiment the method involves annealing at least two oligonucleotides of about or about 40-50 nucleotides in length with an anchor strand about 40-60 nucleotides in length. In different embodiments the anchor strand can utilize 4-6 or 6 degenerate oligonucleotides.

In another embodiment the method involves annealing at least two oligonucleotides of about or about 40-50 nucleotides in length with an anchor strand about 45-55 nucleotides in length. In different embodiments the anchor strand can utilize 4-6 or 6 degenerate oligonucleotides.

In any of these embodiments the method can produce a dsDNA molecule with an error rate of less than one error per 5,300 base pairs.

### Example 1—Hierarchal Synthesis

This example shows the synthesis of a dsDNA molecule of desired sequence having a 100 base pair variable region in a hierarchal method.

The “L0” ligation reaction included two oligonucleotides O1 and O2 (each 45 nucleotides), each of which had a variable sequence of 5 nucleotides, a conserved flanking sequence of about 20 nucleotides, and a primer binding site of about 20 nucleotides. The anchor strand O3 was programmed to have a variable sequence of 10 nucleotides and be 50 nucleotides in length. The oligonucleotides were selected so that the sequence produced by the O1-O3 synthesis (L0) would contain a 10 nucleotide variable sequence that would be a portion of the 100 nucleotide variable sequence of the pre-determined total dsDNA molecule, and would have a variable sequence of about 10 nucleotides. The oligonucleotides were also selected to encode a restriction site for BsaI (a Type IIS endonuclease) on the 5′ side of the DNA molecule (for later ligation with a paired dsDNA molecule having an active recognition site on the 3′ side of the DNA molecule).

A solution was prepared containing oligonucleotides O1-O2 (two oligonucleotides) and O3 (the anchor strand) (2 ul of pool at 100 pM). The oligonucleotides were placed into wells containing T4 DNA ligase buffer (0.5 ul), water (2.4 ul), and T4 DNA ligase (0.1 ul). The solution was incubated for 1 hour at 16° C., then for 10 minutes at 65° C.

After a step of ligation (L0) with T4 DNA ligase a step of PCR amplification (PCR1) was performed using water (2 ul), tailed 5′ and 3′ primers (1 ul, 1 uM) directed to the conserved primer binding sites, a high fidelity thermostable DNA polymerase (5 ul) (Q5U®) (New England Biolabs, Inc., Ipswich, MA), and the L0 reaction product. The PCR protocol was as follows: 98° C. for 30 secs, then 30 cycles of 98° C. (10 secs), 50° C. (10 secs) and 65° C. (15 secs). An enzymatic purification was performed by adding 2 μL of 10-fold diluted stock of Calf-Intestinal Phosphatase (CIP)+Exonuclease I (“CE”) and incubated for 10 minutes at 37 C. 10-fold diluted Proteinase K (2 uL) was added and then incubated for 15 minutes at 37° C. then 10 minutes at 95° C. A purified 98 bp product was confirmed on a gel using a 4% EX E-Gel® (ThermoFisher Corp., Waltham, MA). The product had a variable sequence of 10 nucleotides.

A digestion and ligation step (DL1) was then performed. Water (2.3 ul), T4 ligation buffer (0.5 ul), BsaI enzyme (0.1 ul), T4 DNA ligase (0.1 ul), and the PCR1 product were mixed together. An additional dsDNA fragment having a variable sequence overhang and a 4 bp overlap with the variable sequence of the first dsDNA molecule was added from a parallel PCR1 synthesis reaction. The additional dsDNA fragment can be derived from, for example, a dsDNA molecule with a recognition site on the opposite side of the dsDNA molecule. The mixture was incubated for 1 minute at 37° C. followed by 1 minute at 16° C. and cycled 10 times. Finally, the mixture was held at 80° C. for 20 minutes. A step of PCR (PCR2) was then performed on the DL1 product in a mixture of water (2 ul), 5′ and 3′ primers (1 uM), and DNA polymerase (5 ul), and then diluted 150×. PCR cycles and CIP+CE and proteinase K were performed as above. The dsDNA molecule produced had a variable sequence of 16 nucleotides.

Another digestion and ligation step (DL2) was performed using 2.3 ul water, 10×T4 ligation buffer (0.5 ul), BsaI (0.1 ul), T4 DNA ligase (0.1 ul), and 2 ul of the PCR2 product. An additional dsDNA fragment having a variable sequence overhang and a 4 bp overlap with the first dsDNA molecule was added from a parallel PCR2 synthesis reaction. The mixture was incubated for 1 minute at 37° C. followed by 1 minute at 16° C. and cycled 10 times. Finally the mixture was held at 80° C. for 20 minutes. A step of PCR (PCR3) was then performed on the DL2 product in a mixture of water (2 ul), 5′ and 3′ primers (1 uM), the DNA polymerase above (5 ul), and then diluted 150×. PCR cycles and calf intestinal phosphatase (CE) and proteinase K digestions were performed as above. The dsDNA molecule produced had a variable sequence of 28 nucleotides.

A digestion reaction was performed and the resulting dsDNA fragment was combined with dsDNA fragments from two additional parallel reactions, one of which was a reaction that yielded two dsDNA fragments that were all variable sequence and derived from digestion of a dsDNA molecule with three restriction recognition sites, thus yielding two variable sequences without flanking sequences (e.g. 125, 130 in FIG. 1B). The third additional parallel reaction was performed to maintain the conserved flanking sequence from the opposing (3′) end to allow for efficient ligation and to enable universal primers to be used in downstream PCR (e.g., O12 in FIG. 1B). Amplification products were verified on a gel showing the presence of 88, 68, 68, and 88 bp products.

A ligation step was performed on the dsDNA fragments (DL3) using 16.5 ul water, 10×T4 ligation buffer (2.5 ul), BsaI (0.5 ul), T4 DNA ligase (0.5 ul), and 5 ul of the pooled PCR3 product. The mixture was incubated for 1 minute at 37° C. followed by 1 minute at 16° C. and cycled 25 times. Finally the mixture was held at 80° C. for 20 minutes. A step of PCR (PCR4) was then performed on the product in a mixture of water (6 ul), 5′ and 3′ primers (2 ul of 1 uM), the DNA polymerase above (10 ul), and 2 ul of the digestion and ligation product. PCR cycles and CE and proteinase K were performed as before. Amplification products were verified on a gel showing the presence of a 180 bp product. The molecule was sequenced and found to have the correct sequence, including a 100 nucleotide variable sequence with no errors.

### Example 2—Oligo Library

This example shows construction of a universal oligonucleotide library. Considerations in selecting a library included whether flanking sequences that would serve as robust universal priming sequences and ensure that 5′ and 3′ flanking sequences were distinct enough so that PCR primer sequences would not cross-react in the PCR steps. A common feature in all the flanks was a Type IIS site and this was held constant within the flanking sequence and designed around. These sequences were generated by computational design but can also be generated manually.

Different flanking sequences were empirically selected by using approximately eight sequences and testing them directly in PCR. The best performing flanking sequence set based on empirical data was then selected. The “flank set” was tested with the 5′ and 3′ primer pair, the 5′ only, and the 3′ only to ensure that the expected PCR product would be generated.

After selecting the flanking sequences, the variable sequences were added to the sequences. Note that all possible permutations of the variable bases were needed to be able to construct a library that could synthesize any possible DNA sequence. For example, if five variable bases were added to the 3′ end of O1, there was 4 to the 5^{th} power or 1,024 different O1 sequences in separate microtiter wells where 4 is the number of DNA bases available and 5 is the number of variable bases utilized in the O1 oligo. These variable sequences were generated by available computational design programs but can also be generated manually.

In the case of O1, five variable bases were added to the 3′ end. In the case of O2, five variable bases were added to the 5′ end. In the case of O3, the variable sequence containing four non-degenerate bases were added to the central part of the oligo to support the ligation of O1 and O2 at their abutting interfaces and then surrounded by three degenerate N bases on each side as these bases prevent the unnecessary expansion of the library. The degenerate N bases were synthesized on the oligo synthesizer by combining all four DNA bases for the N position, thus a O3 anchor oligo was a mixture of sequences. The O3 anchor oligo had a total of six N positions and thus a total 4 to the 6^{th} power or 4,096 different molecules within a single well of the library. Not all the molecules in this library well were viable 03 anchors for the ligation of O1+O2, but only a fraction of the 4,096 molecules were needed to support a robust L0 ligation.

The oligos that made up the library were then synthesized in microtiter plate format in such a way that all oligo members had a discrete well location within the library. The wells were in single micro-tubes or microtiter plate formats of 96 and 384-wells, but they can be any format that allows for the physical separation of library oligo members. The location of each member was precisely known and could be accessed when the oligo components were pooled together, either manually or by laboratory liquid handling automation.

When synthesizing a sequence, for example a 100 bp sequence that is a portion of a specific gene, the following steps were followed:

Three oligos (O1, O2 & O3) were pooled into a single well and these oligos corresponded to the first 10 bp (bases 1 to 10) of the 100 bp variable sequence to be synthesized in this example.

Three more oligos were then pooled (i.e., the next set of O1, O2 & O3) into an adjacent well. These oligos constituted another 10 bp of the variable sequence but overlapped the first set of oligos above by 4 bp, which constituted bases 6-14 of the 100 bp sequence in this example.

This process was repeated until there were enough starting pools to make the entire DNA molecule having the 100 bp variable sequence. In this example, there were a total of 16 starting pools, the sequence of each pool overlapping the next by 4 bp. After all the pools were established in the reaction wells, the process of synthesis was started.

Table 1: This table shows the number of oligo members in an entire library set that were needed to build any DNA molecule having the variable sequence of 10→16→28→100 bps. The total number of library members needed was 9,216.

| | O1 | O2 | O3 | O1 | O2 | O3 | |
|---|---|---|---|---|---|---|---|
| Library # | Assembly 1 | Assembly 1 | Assembly 1 | Assembly 2 | Assembly 2 | Assembly 2 | Total |
| 1 | 1024 | 1024 | 256 | 1024 | 1024 | 256 | 4608 |
| 2 | 1024 | 1024 | 256 | 1024 | 1024 | 256 | 4608 |
| | | | | | | Grand Total --> | 9216 |

Table 2: This table shows the nucleotide lengths for each of the oligo members in the library set. The length of the non-degenerate nucleotides of the variable sequence is shown in parenthesis.

| | O1 | O2 | O3 | O1 | O2 | O3 |
|---|---|---|---|---|---|---|
| Library # | Assembly 1 | Assembly 1 | Assembly 1 | Assembly 2 | Assembly 2 | Assembly 2 |
| 1 | 45(5) | 45(5) | 50(4) | 45(5) | 45(5) | 50(4) |
| 2 | 45(5) | 45(5) | 50(4) | 45(5) | 45(5) | 50(4) |

### Example 3—Preparation for Assembly of SARS CoV-2 Spike Gene

This example shows the assembly of 72 dsDNA molecules having overlapping 100 base pair variable sequences in a hierarchal method for the synthesis of an approximately 4 kb SARS-CoV-2 spike protein. The 100 bp variable sequences in each dsDNA molecule are sub-sequences of the spike gene of SARS-CoV-2. The dsDNA molecules containing the sub-sequences were synthesized as in Example 1 to yield seventy-two 180 bp product sequences having 100 bp variable sequences that overlapped by about 4 bp. In the PCR4 step the dsDNA molecules were biotinylated using biotinylated primers and standard methods, and then combined into a single pool.

DNA Capture and Release of 100 bp Fragments (with Flank Removal)

DNA microbeads (spherical particles having a silicon core and covered with a layer of paramagnetic material) were prepared and used according to manufacturer's instructions (Dynabeads®, Life Technologies, Oslo, Norway).

The new microbeads were resuspended in a vial and vortexed for about 30 seconds or tilted and rotated for 5 min. The microbeads were transferred (50 ul of beads/sample) to a centrifuge tube containing the pooled PCR4 products. One ml of 1× bind and wash (B&W) buffer was added to the tube, and the tube vortexed for 5 sec. The tube was placed on a magnet for 1 min to bind the DNA and the supernatant was discarded. The tube was removed from the magnet and the washed beads resuspended in at least 1 ml of washing buffer, or in the initial volume of microbeads taken from the initial vial. This was repeated for a total of two washes. The microbeads were resuspended in 2×B&W buffer with 2× volume of the original beads (e.g. 100 ul beads stock to 200 ul 2×B&W).

An equal volume of the biotinylated DNA (such as 50 ul PCR4 pool+50 ul prewashed beads) was added to dilute the NaCl concentration in the 2×B&W buffer from 2 M to 1 M for optimal binding and immobilization. The sample was incubated for about 30 minutes at room temperature using gentle rotation. The beads were then captured on the magnet for 2 min, and washed with 1×B&W buffer. This was repeated for a total of three washes.

The captured beads were resuspended in 1×NEB3 Buffer (1×BsmBI buffer, dilution of 10×r3.1 buffer with ddH2O), with the same volume as the PCR pool used. 2 ul of Type II enzyme (BsmBI) were added per 50 ul of volume and the beads resuspended and incubated for 60 min at 55° C., then cooled to room temperature. The beads were captured on the magnet for 2-3 min and the liquid digestion transferred to a new tube or well that contained the pool of released 100 bp fragments.

The PCR4 pool digest was used as a template to perform polymerase chain assembly (PCA) with 30 cycles to assemble the final dsDNA molecule. While various cycles can be utilized, the PCA cycling parameters were as follows: 1. 98° C., 1 min; 2. 98° C., 30 sec; 3. 72° C., 30 sec (add 15 s/cycle); 4. 65° C. for 1 min (add 15 sec/cycle); 5. 60° C. for 1 min; 6. 55° C. for 45 s, 7. cycle back to 2. for 29 more cycles, finally 8. 72° C. for 5 minutes, and 9. store at 10° C.

5 ul of the PCA reaction product was added to 20 ul of PCR master mix containing primers matching the 5′ and 3′ ends of the spike gene sequence, and PCR was performed with 30 cycles according to the following cycle parameters: 1. 98° C. for 1 min; 2. 98° C. for 30 seconds, 3. 72° C. for 45 sec (add 15 sec/cycle); 4. 60° C. for 1 min (add 15 sec/cycle); 5. cycle back to 2. nine times; 6. Then 98° C. for 30 sec; 7. 72° C. for 3 min; 8. 65° C. for 4 min; 9. cycle back to step 6 nineteen more times; then 10. 72° C. for 5 min; 11. store at 10° C.

As shown in FIG. 7, a 3,942 bp product was obtained. The identity of the full length gene was confirmed as SARS-Cov2 spike gene by cloning and DNA sequencing analysis and determined to have an error rate of approximately one error per 5,400 bp prior to applying an enzymatic error correction step.

Sequences

SEQ ID NO: 1, DNA, artificial sequence
AGGGA
SEQ ID NO: 2, DNA, artificial sequence
CGTTG
SEQ ID NO: 3, DNA, artificial sequence
NNNACTCNNN
SEQ ID NO: 4, DNA, artificial sequence
TTGCG
SEQ ID NO: 5, DNA, artificial sequence
TAGCG
SEQ ID NO: 6, DNA, artificial sequence
NNNTACGNNN
SEQ ID NO: 7, DNA, artificial sequence
AGGGAGTTGC
SEQ ID NO: 8, DNA, artificial sequence
TTGCGTAGCG
SEQ ID NO: 9, DNA, artificial sequence
AGGGAG
SEQ ID NO: 10, DNA, artificial sequence
TTGC
SEQ ID NO: 11, DNA, artificial sequence
GCAACTCCCT
SEQ ID NO: 12, DNA, artificial sequence
TTGCGTAGCG
SEQ ID NO: 13, DNA, artificial sequence
CGCTAC
SEQ ID NO: 14, DNA, artificial sequence
GCAA
SEQ ID NO: 15, DNA, artificial sequence
AGGGAGTTGCGTAGCG
SEQ ID NO: 16, DNA, BsmBI recognition site, Bacillus stearothermophilus
CGTCTC(N)
SEQ ID NO: 17, DNA, Bsal recognition site, Bacillus stearothermophilus
GGTCTC(N)

Although the invention has been described with reference to the presently preferred embodiment, it should be understood that various modifications can be made without departing from the spirit of the invention. Accordingly, the invention is limited only by the following claims.

1. A method of synthesizing a DNA molecule having a desired sequence comprising:
a) annealing at least two oligonucleotides to an anchor strand so that the at least two oligonucleotides annealed to the anchor strand abut one another on the anchor strand;
wherein the at least two oligonucleotides each comprise a primer binding site on a 3′ or 5′ end, and a variable sequence on the opposing 5′ or 3′ end, and a conserved flanking sequence in between the primer binding site and the variable sequence; and
wherein the anchor strand comprises conserved flanking sequences complementary to those on the at least two oligonucleotides, and further comprises at least one variable sequence, wherein at least a portion of the at least one variable sequence on the anchor strand is complementary to at least a portion of the variable sequences on the at least two oligonucleotides;
b) ligating the at least two oligonucleotides annealed to the anchor strand to produce a first dsDNA molecule;
c) performing an amplification step on the first dsDNA molecule having a desired sequence and comprising a conserved flanking sequence inside each of the 3′ and 5′ ends, and a variable sequence inside the conserved flanking sequences.

2. The method of claim 1 further comprising contacting the first dsDNA molecule with a restriction endonuclease to produce first dsDNA fragments comprising 3′ and/or 5′ overhang sequences comprising a portion of the variable sequence from the first dsDNA molecule, providing at least one first additional dsDNA fragment comprising a 3′ and/or 5′ overhang sequence that is at least partially complementary to an overhang sequence of at least one of the first dsDNA fragments;
annealing the first dsDNA fragments and at least one first additional dsDNA fragment by the 3′ and/or 5′ overhang sequences; and
ligating the annealed dsDNA fragments to produce a second dsDNA molecule comprising a conserved flanking sequence inside each of the 3′ and 5′ ends, and a variable sequence inside the 3′ and 5′ conserved flanking sequences that is longer than the variable sequence on the first dsDNA molecule.

3. The method of claim 2 wherein the at least one first additional dsDNA fragment is the product of a parallel DNA synthesis reaction, further wherein the first dsDNA molecule has a recognition site for a restriction endonuclease on the 5′ or 3′ side of the molecule, and the first additional dsDNA fragment is derived from restriction cleavage of a dsDNA molecule having a recognition site for a restriction endonuclease on the opposing 3′ or 5′ side of the molecule.

4. The method of claim 3 further comprising contacting the at least one second dsDNA molecule with a restriction endonuclease to produce a plurality of second dsDNA fragments comprising 3′ and/or 5′ overhang sequences and a conserved flanking sequence inside each of the 3′ or 5′ ends;
providing at least one second additional dsDNA fragment comprising a 3′ and/or 5′ overhang sequence that is at least partially complementary to an overhang sequence of at least one of the second dsDNA fragments;
annealing the plurality of second dsDNA fragments to the at least one second additional dsDNA fragment by the 3′ and/or 5′ overhang sequence(s); and
performing a step of ligation to produce a third dsDNA molecule comprising a conserved flanking sequence on the 3′ and 5′ ends, and a variable sequence inside the conserved flanking sequences that is longer than the variable sequence of the second dsDNA molecule.

5. The method of claim 4 wherein the at least one second additional dsDNA fragment is the product of a parallel DNA synthesis reaction, further wherein the second dsDNA molecule has a recognition site for a restriction endonuclease on the 5′ or 3′ side of the molecule, and the second additional dsDNA fragment is derived from restriction cleavage of a dsDNA molecule having a recognition site for a restriction endonuclease on the opposing 3′ or 5′ side of the molecule.

6. The method of claim 5 further comprising reacting the at least one third dsDNA molecule with a restriction endonuclease to produce a plurality of third dsDNA fragments comprising 3′ and/or 5′ overhang sequences and a conserved flanking sequence inside each of the 3′ or 5′ ends;
providing at least one third additional dsDNA fragment comprising a 3′ and/or 5′ overhang sequence that is at least partially complementary to an overhang sequence of at least one of the third dsDNA fragments;
annealing the plurality of third dsDNA fragments to the at least one third additional dsDNA fragment by the 3′ and/or 5′ overhang sequences; and
performing a step of ligation to produce a fourth dsDNA molecule comprising a conserved flanking sequence on the 3′ and 5′ ends, and a variable sequence inside the conserved flanking sequences that is longer than the variable sequence of the third dsDNA molecule.

7. The method of claim 6 wherein the at least one third additional dsDNA fragment is the product of a parallel DNA synthesis reaction, further wherein the third dsDNA molecule has a recognition site for a restriction endonuclease on the 5′ or 3′ side of the molecule, and
the first additional dsDNA fragment is derived from restriction cleavage of a dsDNA molecule having a recognition site for a restriction endonuclease on the opposing 3′ or 5′ side of the molecule.

8-12. (canceled)

13. The method of claim 7 wherein the first dsDNA molecule comprises a variable sequence of 8-12 base pairs.

14. The method of claim 7 wherein the paired dsDNA molecule comprises a variable sequence of 8-12 base pairs.

15. The method of claim 7 wherein the second dsDNA molecule comprises a variable sequence of 14-18 base pairs.

16. The method of claim 7 wherein the third dsDNA molecule comprises a variable sequence of 24-32 base pairs.

17. The method of claim 7 wherein the fourth dsDNA molecule comprises a variable sequence of 90-110 base pairs.

18. The method of claim 7 wherein the at least two oligonucleotides have a variable sequence of 4-6 nucleotides.

19. The method of claim 7 wherein the amplification step is performed by the polymerase chain reaction (PCR).

20. The method of claim 7 wherein the variable sequence of the anchor strand is equal in length to the lengths of the variable sequences on the at least two oligonucleotides.

21. The method of claim 7 wherein the anchor strand comprises a variable sequence present in between the two sequences complementary to the conserved flanking sequences on the at least two oligonucleotides.

22. The method of claim 7 wherein the at least two oligonucleotides bound to the anchor strand abut one another on the anchor strand at their variable sequences.

23. The method of claim 7 wherein the portion of the variable sequence on the anchor strand that is complementary to the conserved flanking sequence on the at least two oligonucleotides comprises 2-6 nucleotides or 14-18 nucleotides or 26-30 nucleotides or 90-110 nucleotides.

24. The method of claim 7 wherein the at least two oligonucleotides and anchor strand further comprise a recognition site for a restriction endonuclease.

25. The method of claim 24 wherein the restriction endonuclease is a Type IIS endonuclease.

26. The method of claim 7 wherein the step of ligation occurs spontaneously.

27. The method of claim 7 wherein the anchor strands comprise 4-6 degenerate nucleotides.

28. The method of claim 27 wherein the degenerate nucleotides comprise a universal or randomized base.

29. The method of any one of claims 1-29 claim 7 wherein the DNA molecule of desired sequence has an error rate of less than 1 base pair per 2,000 versus the desired sequence.

30. (canceled)

31. The method of claim 7 wherein the DNA molecule of desired sequence is assembled from a library of fewer than 20,000 or 10,000 members.

32. The method of claim 7 wherein the primer binding sites comprise universal primer binding sites.

33. The method of claim 7 wherein the product DNA molecule is up to 4,000 bp or up to 5,000 bp in length.

34-48. (canceled)
